(19) United States
(12) Patent Application Publication
(10) Pub. No.: US 2025/0296906 A1
(43) Pub. Date: Sep. 25, 2025

(54) METHOD FOR PREPARING 4-HYDROXY-2-METHYLENE-BUTANAL, 4-HYDROXY-2-METHYL-BUT-2-ENAL AND ESTERS THEREOF

(71) Applicant: BASF SE, Ludwigshafen am Rhein (DE)

(72) Inventors: Bernd SCHAEFER, Ludwigshafen am Rhein (DE); Wolfgang SIEGEL, Ludwigshafen am Rhein (DE)

(21) Appl. No.: 19/105,058

(22) PCT Filed: Aug. 23, 2023
(86) PCT No.: PCT/EP2023/073157

§ 371 Date: Feb. 20, 2025

(30) Foreign Application Priority Data
Aug. 24, 2022 (EP) 22191946.7
Aug. 24, 2022 (EP) 22191954.1

(51) Int. Cl. C07C 45/33; B01J 31/02; B01J 35/39; C07C 409/04

(52) U.S. Cl. CPC C07C 45/33; B01J 31/0244; B01J 35/39; C07C 409/04; B01J 2231/766

(57) ABSTRACT

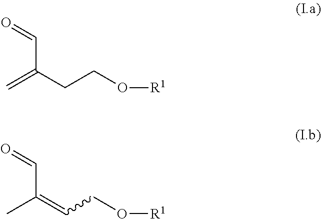

The present invention relates to a method for preparing 4-hydroxy-2-methylene-butanal, 4-hydroxy-2-methyl-but-2-enal and esters thereof of the formula (I.a) and (I.b) where R¹ is as defined in the claims and the description, by subjecting isoprenol or an ester thereof to a photooxidation in the presence of a photosensitizer and an acylating agent. The invention relates moreover to certain hydroperoxides of the compounds (I.a) or (I.b), and to the use thereof as intermediates in the synthesis of compounds (I.a) and (I.b) or in the synthesis of retinol, stereoisomers and derivatives thereof.

The present invention relates to a method for preparing 4-hydroxy-2-methylene-butanal, 4-hydroxy-2-methyl-but-2-enal and/or esters thereof of the formula I.a and I.b as defined below by subjecting isoprenol or an ester thereof of the formula II.a as defined below to a photooxidation in the presence of a photosensitizer and an acylating agent. The invention relates moreover to the use of certain compounds of the formula I.a or I.b as defined below as intermediates in the synthesis of retinol, stereoisomers and derivatives, in particular esters, thereof; to certain hydroperoxides of the formula III.a, III.b or III.c as defined below; and to the use thereof as intermediates in the synthesis of compounds I.a and I.b or in the synthesis of retinol, stereoisomers and derivatives, in particular esters, thereof.

### TECHNICAL BACKGROUND

4-Acetoxy-2-methylbut-2-enal (the E isomer of which is also called C₅ acetate), the acetic acid ester of 4-hydroxy-2-methyl-but-2-enal mentioned above, is an important building block in industrial syntheses of retinol, stereoisomers and derivatives thereof. Acetoxy-2-methylbut-2-enal, for example in form of its E-isomer C₅ acetate, is currently obtained on industrial scale from vinylglycol-1,2-diacetate (VGDA), a side product from an industrial process, via hydroformylation and deacetoxylation. The latter steps are described, for example, in DE 10117065 and the references cited therein.

The economic availability of VGDA is however dependent on the unaltered continuation of the industrial process from which it stems. Given the increasing unpredicatbility of the lifespan of such processes, be it because of increasing costs for raw materials and energy or ecological demands or increasingly unreliable supply chains, it is desirable to have alternative routes towards C₅ acetate, isomers and derivatives thereof at hand. Also the limited quantities of VGDA available by said process make alternative routes desirable.

Other known synthetic pathways towards C₅ acetate are the oxidation of prenyl acetate with selenium dioxide, as described, for example, in CN 108997112, the oxidation of benzly prenyl ether, as described, for example, by S. Inoue et al in Chemistry Lett. 1986, 2035-2038, the oxidation of prenyl chloride with oxygen, as described, for example, in CN 108707076, the oxidation of isoprene, as described, for example, by P. A. Wehrli et al., Synthesis, 1977, 649-650, the acetylation of prenol and the oxidation of the resulting prenol acetate with peroxides under irradiation in the presence of a photosensitizer, as described in CN 110981724 A, and the oxidation of prenol acetate in an electrochemical process, as described in CN 111270261 A. These routes are however not suitable for an application on industrial scale.

4-Acetoxy-2-methylbut-2-enal, the basic alcohol 4-hydroxy-2-methyl-but-2-enal and other esters thereof can be obtained from the respective 2-methylene double bond isomer (i.e. from 3-formylbut-3-enyl acetate, 4-hydroxy-2-methylene-butanal or other esters thereof) by known methods, for example via Pd-catalyzed C—C double bond isomerization as described e.g. in U.S. Pat. No. 4,124,619 or CN 103467287.

It is desirable to find an alternative route to 4-hydroxy-2-methyl-but-2-enal or 4-hydroxy-2-methylene-butanal and esters of these alcohols; ideally, this route should be suitable for an industrial scale. Out of environmental and economic reasons, this route, at least the essential steps thereof, should in particular also work with very low amounts of solvents; ideally neat, i.e. in the absence of solvents.

Isoprenol (3-methylbut-3-en-1-ol) is a bulk chemical readily available from isobutene and formaldehyde. Double bond isomerization thereof leads to prenol (3-methylbut-2-en-1-ol). Esters thereof are obtainable by standard esterification processes.

Photooxidation of alkenes with singlet oxygen to allylic hydroperoxides (Schenck ene reaction) and subsequent dehydration to α-enones have been described in the art.

E. D. Mihelich et al. describe in J. Org. Chem. 1983, 48, 4135-4137 the preparation of α-enones by the reaction of cycloalkenes, methyl oleate and other olefinically unsaturated hydrocarbons with singlet oxygen. To this purpose, oxygen is passed through a reaction mixture containing the olefinically unsaturated hydrocarbon, acetic anhydride, pyridine, N,N-dimethylaminopyridine (DMAP) and tetraphenylporphyrin (TPP) as photosensitizer in methylene chloride, and the reaction is simultaneously irradiated with a sodium vapor lamp.

H.-J. Liu et al. describe in Tetrahedron Lett. 1993, 34 (28), 4435-4438 the synthesis of (+)-Qinghaosu. The synthesis encompasses inter alia a step where a tricyclic olefinically unsaturated carbocyclic ring is converted into the corresponding α-enone via irradiation of a reaction mixture containing the unsaturated ring, acetic anhydride, pyridine, DMAP and TPP in methylene chloride through which oxygen is passed.

K. You et al. describe in Journal of Photochemistry and Photobiology A: Chemistry, 2011, 217, 321-325 the photosensitized oxidation of α-pinene, β-pinene and limonene inter alia to α-enones using tetrachlorotetraiodo-fluorescein sodium salt as sensitizer in methanol or DMF as solvents in the presence or absence of lutidine and/or acetic anhydride.

P. Bayer et al. describe in Green Chem., DOI: 10.1039/d0gc00436g the photooxygenation of alkenes with singlet oxygen to hydroperoxides in a solvent-free continuous-flow reaction set-up. The further conversion of the labile hydroperoxides to, for example, α-enones, is described schematically as reaction of the hydroperoxide with acetic anhydride and pyridine in dichloromethane.

E. L. Clennan et al. describe in Photochemistry and Photobiology, 2006, 82, 1226-1232 the photooxidation of various allylic alcohols with singlet oxygen. Inter alia, prenol is converted in the presence of TPP in CDCl₃ to 3-methyl-but-2-enal, 3,3-dimethyloxirane-2-carbaldehyde, 2-hydroperoxy-3-methyl-but-3-en-1-ol and 5,5-dimethyl-1,2-dioxolan-3-ol.

### SUMMARY OF THE INVENTION

The present inventors found that 4-hydroxy-2-methyl-but-2-enal, 4-hydroxy-2-methylene-butanal and esters of these alcohols can be obtained by subjecting isoprenol or an ester thereof to a photooxidation in the presence of a photosensitizer and an acylating agent, or by subjecting isoprenol or an ester thereof to a photooxidation in the presence of a photosensitizer and subsequently reacting the hydroperoxides formed in the photooxidation with an acylating agent.

The invention thus relates to a method for preparing a compound of the formula I.a or of the formula I.b or a stereoisomer of the compound I.a or I.b or a mixture of different stereoisomers of the compounds I.a and/or I.b or a mixture of different compounds I.a and/or I.b

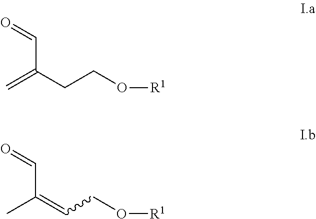

wherein
R¹ is hydrogen or —C(═O)R²; and
R² is C₁-C₂₀-alkyl;
which method comprises
(i) providing a reaction mixture comprising a compound of the formula II.a

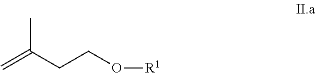

wherein R¹ is as defined above;
a photosensitizer and optionally an acylating agent;
(ii) passing an oxygen-containing gas through the reaction mixture provided in step (i) and simultaneously irradiating the reaction mixture with light;
(iii) if in step (i) no acylating agent has been provided, adding an acylating agent to the reaction mixture obtained in step (ii);
(iv.1) if desired, after completion of the reaction (to the desired degree), isolating the one or more compounds (I.a) or (I.b) obtained in step (ii) or (iii); and
(v.1) if desired hydrolysing the one or more compounds I.a or I.b isolated in step (iv.1) to compounds I.a or I.b wherein R¹ is hydrogen;
   or
(iv.2) if desired, hydrolysing the reaction mixture obtained in step (ii) or (iii); and
(v.2) if desired, isolating the one or more compounds I.a or I.b obtained in step (iv.2).

The invention relates moreover to the use of the compound of the formula I.a or I.b different from (E)-4-acetoxy-2-methylbut-2-enal or of a stereoisomer of the compound I.a or I.b different from (E)-4-acetoxy-2-methylbut-2-enal or of a mixture of different stereoisomers of the compound I.a and/or I.b or of a mixture of different compounds I.a and/or I.b as defined above as intermediates in the synthesis of retinol, stereoisomers thereof, derivatives thereof (where the derivatives are in particular esters thereof) or stereoisomers of derivatives thereof (where the derivatives are in particular esters thereof).

The invention relates also to a hydroperoxide compound of the formula III.a, III.b or III.c or a stereoisomer of the compound of the formula III.a, III.b or III.c or a mixture of different stereoisomers of the compound III.a, III.b and/or III.c or a mixture of different compounds III.a, III.b and/or III.c

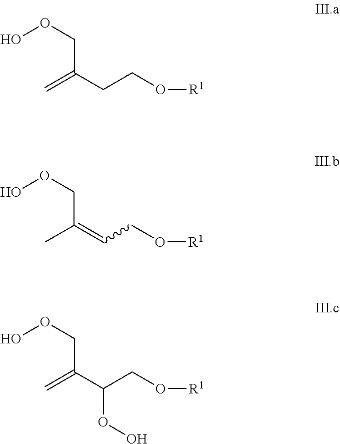

where
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl;

preferably to a hydroperoxide compound of the formula III.a or III.b or a stereoisomer of the compound of the formula III.a or III.b or a mixture of different stereoisomers of the compound III.a and/or III.b or a mixture of different compounds III.a and/or III.b

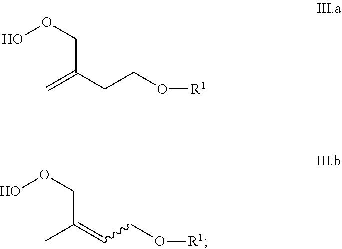

and to the use of said hydroperoxides of the formula III.a, III.b or III.c or of a stereoisomer of the compound of the formula III.a, III.b or III.c or of a mixture of different stereoisomers of the compound III.a, and/or III.b and/or III.c or of a mixture of different compounds III.a, III.b and/or III.c as defined above, where however in compound III.b R¹ can also be hydrogen, preferably of said hydroperoxides of the formula III.a or III.b or of a stereoisomer of the compound of the formula III.a or III.b or of a mixture of different stereoisomers of the compound III.a and/or III.b or of a mixture of different compounds III.a and/or III.b as defined above, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (where the derivatives are preferably esters thereof (i.e. retinol esters), retinal or retinoic acid, and are in particular esters thereof) or stereoisomers of derivatives thereof (where the derivatives are preferably esters thereof (i.e. retinol esters), retinal or retinoic acid, and are in particular esters thereof).

### DETAILED DESCRIPTION OF THE INVENTION

### Definitions

“Alkyl” is used in the usual sense. The term “alkyl” refers to saturated straight-chain (linear) or branched hydrocarbon radicals having 1 or 2 (“C₁-C₂-alkyl”), 1 to 4 (“C₁-C₄-alkyl”) or 1 to 20 (“C₁-C₂₀-alkyl”) carbon atoms. C₁-C₂-Alkyl denotes a saturated linear or branched aliphatic acyclic hydrocarbon radical with 1 or 2 carbon atoms. Examples are methyl and ethyl. C₁-C₄-Alkyl denotes a saturated linear or branched aliphatic acyclic hydrocarbon radical with 1 to 4 carbon atoms. Examples are methyl, ethyl, n-propyl, isopropyl, n-butyl, sec-butyl, isobutyl and tert-butyl. C₁-C₂₀-Alkyl denotes a saturated linear or branched aliphatic acyclic hydrocarbon radical with 1 to 20 carbon atoms. Examples are, in addition to those mentioned for C₁-C₄-alkyl, n-pentyl, 1-methylbutyl, 2-methylbutyl, 3-methylbutyl, 2,2-dimethylpropyl, 1-ethylpropyl, 1,1-dimethylpropyl, 1,2-dimethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, 2-propylheptyl, n-undecyl, n-dodecyl, n-tridecyl, n-tetradecyl, n-pentadecyl, n-hexadecyl, n-heptadecyl, n-octadecyl, n-nonadecyl, n-eicosyl and (other) structural isomers thereof. n-C₁₅-Alkyl is CH₃(CH₂)₁₄—.

Chlorinated C₁-C₂-alkanes are methane or ethane in which a part or all of the hydrogen atoms are replaced by chlorine atoms. Examples are dichloromethane (methylene chloride), trichloromethane (chloroform), tetrachloromethane (carbon tetrachloride), 1,1-dichloroethane, 1,2-dichloroethane, 1,1,1-trichloroethane, 1,1,2-trichloroethane, 1,1,1,2-tetrachloroethane, 1,1,2,2-tetrachloroethane and pentachloroethane.

The term “stereoisomers” as used in context with the present invention relates to optical isomers, such as enantiomers or diastereomers, the latter existing due to more than one stereogenic center in the molecule, but in particular to Z/E isomers (due to the presence of correspondingly substituted double bonds or ring systems). Thus, stereoisomers of the compounds I.b are primarily the E isomer (E)-I.b and the Z isomer (Z)-I.b:

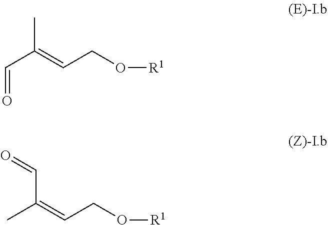

Optical isomers of compounds I.b occur if the radical R¹ is —C(═O)R², and R² is a C₄-C₂₀-alkyl group having one or more stereogenic centers, such as in sec-butyl.

Analogously, stereoisomers of the compounds III.b are primarily the E isomer (E)-III.b and the Z isomer (Z)-III.b:

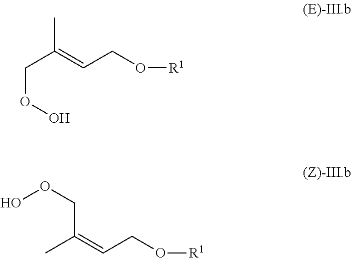

Optical isomers of compounds III.b occur if the radical R¹ is —C(═O)R², and R² is a C₄-C₂₀-alkyl group having one or more stereogenic centers, such as in sec-butyl.

Analogously, optical isomers of compounds I.a occur if the radical R¹ is —C(═O)R², and R² is a C₄-C₂₀-alkyl group having one or more stereogenic centers, such as in sec-butyl; and optical isomers of compounds III.a and III.c occur if the radical R¹ is —C(═O)R², and R² is a C₄-C₂₀-alkyl group having one or more stereogenic centers, such as in sec-butyl.

Mixtures of different stereoisomers of the compounds I.b are primarily mixtures of the E- and the Z-isomer, but can also be mixtures of enantiomers or diastereomers of compounds I.b in which R¹ is —C(═O)R², and R² is a C₄-C₂₀-alkyl group having one or more stereogenic centers. Analogously, mixtures of different stereoisomers of the compounds III.b are primarily mixtures of the E- and the Z-isomer, but can also be mixtures of enantiomers or diastereomers of compounds III.b in which R¹ is —C(═O)R², and R² is a C₄-C₂₀-alkyl group having one or more stereogenic centers. Mixtures of different stereoisomers of the compounds I.a are mixtures of enantiomers or diastereomers of compounds I.a in which R¹ is —C(═O)R², and R² is a C₄-C₂₀-alkyl group having one or more stereogenic centers. Analogously, mixtures of different stereoisomers of the compounds III.a are mixtures of enantiomers or diastereomers of compounds III.a in which R¹ is —C(═O)R², and R² is a C₄-C₂₀-alkyl group having one or more stereogenic centers. Analogously, mixtures of different stereoisomers of the compounds III.c are mixtures of enantiomers or diastereomers of compounds III.c in which R¹ is —C(═O)R², and R² is a C₄-C₂₀-alkyl group having one or more stereogenic centers.

Mixtures of the compounds I.a or I.b can be mixtures of two or more different compounds I.a, the compounds I.a present in the mixture differing in the radical R¹; mixtures of two or more different compounds I.b, the compounds I.b present in the mixture differing in the radical R¹; mixtures of a compound I.a and a compound I.b, where in compounds I.a and I.b the radical R¹ has the same meaning; mixtures of a compound I.a and a compound I.b, where in compounds I.a and I.b the radical R¹ has different meanings; mixtures of a compound I.a with two or more different compounds I.b; mixtures of a compound I.b with two or more different compounds I.a; or mixtures of two or more different compounds I.a with two or more different compounds I.a. Primarily, however, mixtures of the compounds I.a or I.b refers to mixtures of a compound I.a and a compound I.b, where in compounds I.a and I.b the radical R¹ has the same meaning. Compounds I.b in the above-defined mixtures can be present as the pure E isomer, the pure Z isomer or a mixture of the E and Z isomers.

The analogous definition applies to mixtures of the compounds III.a, III.b or III.c.

A photosensitizer in terms of the present invention is an organic molecule (generally a dye) which, when subjected to irradiation (generally to electromagnetic radiation in the UV, in the visible or in the near IR region) can convert triplet oxygen to singlet oxygen: Upon irradiation, the sensitizer forms the corresponding excited singlet state. Intersystem crossing affords the excited triplet state of the sensitizer, thus transferring energy to triplet oxygen to form singlet oxygen.

“Light” in the proper sense is electromagnetic radiation with a wavelength (range) in the visible spectrum (380 to 780 nm). However, in terms of the present invention, unless specified otherwise, the term “light” also encompasses the directly adjacent wavelength spectrum, i.e. near IR (>780 nm to 1 μm) and near UV (315 to <380 nm).

Retinol is (2E,4E,6E,8E)-3,7-dimethyl-9-(2,6,6-trimethylcyclohex-1-enyl)nona-2,4,6,8-tetraen-1-ol (all-trans). Stereoisomers of retinol in terms of the present invention relate to retinol, in which however one, two, three or all four of the double bonds in the 2-, 4-, 6- and 8-position(s) has/have Z geometry. Specific examples for such stereoisomers are: (2Z,4E,6E,8E)-3,7-dimethyl-9-(2,6,6-trimethylcyclohex-1-enyl)nona-2,4,6,8-tetraen-1-ol; (2E,4Z,6E,8E)-3,7-dimethyl-9-(2,6,6-trimethylcyclohex-1-enyl)nona-2,4,6,8-tetraen-1-ol; (2Z,4Z,6E,8E)-3,7-dimethyl-9-(2,6,6-trimethylcyclohex-1-enyl)nona-2,4,6,8-tetraen-1-ol; or (2Z,4E,6Z,8E)-3,7-dimethyl-9-(2,6,6-trimethylcyclohexen-1-yl)nona-2,4,6,8-tetraen-1-ol (also known as (13Z) retinol under carotenoid nomenclature).

Retinol derivatives in terms of the present invention are preferably retinol esters, i.e. retinol in which the —OH group is esterified to a group —O—C(═O)R, where R is an organic moiety, and is preferably R². Retinol derivatives are however also oxidized forms of retinol, such as retinal (—CH₂OH group is oxidized to —CHO) or retinoic acid (—CH₂OH group is oxidized to —C(═O)OH).

Stereoisomers of retinol derivatives are retinol derivatives as defined above, in which however one, two, three or all four of the double bonds in the 2-, 4-, 6- and 8-position(s) has/have Z geometry.

### EMBODIMENTS (E.X) OF THE INVENTION

General and preferred embodiments E.x are summarized in the following, nonexhaustive list. Further preferred embodiments become apparent from the paragraphs following this list.

E.1. A method for preparing a compound of the formula I.a or of the formula I.b or a mixture thereof or a stereoisomer of the compound I.a or I.b or a mixture of different stereoisomers of the compound I.a and/or I.b or a mixture of different compounds I.a and/or I.b

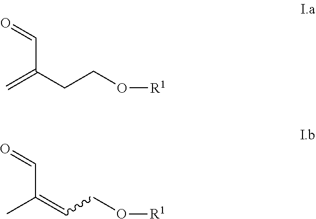

wherein
R¹ is hydrogen or —C(═O)R²; and
R² is C₁-C₂₀-alkyl;
which method comprises
(i) providing a reaction mixture comprising a compound of the formula II.a

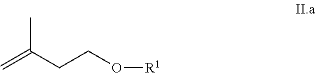

wherein R¹ is as defined above;
a photosensitizer and optionally an acylating agent;
(ii) passing an oxygen-containing gas through the reaction mixture provided in step (i) and simultaneously irradiating the reaction mixture with light;
(iii) if in step (i) no acylating agent has been provided, adding an acylating agent to the reaction mixture obtained in step (ii);
(iv.1) if desired, after completion of the reaction, isolating the one or more compounds (I.a) or (I.b) obtained in step (ii) or (iii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (ii) or (iii); and
(v.2) if desired, isolating the one or more compounds (I.a) or (I.b) obtained in step (iv.2).

E.2. The method according to embodiment E.1, where R² is C₁-C₄-alkyl or n-C₁₅-alkyl.

E.3. The method according to embodiment E.2, where R² is C₁-C₄-alkyl.

E.4. The method according to embodiment E.3, where R² is methyl.

E.5. The method according to any of the preceding embodiments, where in the compound II.a R¹ is —C(═O)R².

E.6. The method according to any of the preceding embodiments, where the acylating agent used in step (i) or (iii) is selected from the group consisting of carboxylic halides R^{2a}—C(═O)—X, carboxylic acid anhydrides R^{2a}—C(═O)—O—C(═O)—R^{2a}, and ketenes R^{2a}—C(H)═C═O, where R^{2a} has independently one of the meanings given for R² in any of embodiments E.1 to E.4 and X is Cl, Br or I.

E.7. The method according to embodiment E.6, where the acylating agent used in step (i) or (iii) is a carboxylic acid anhydride R^{2a}—C(═O)—O—C(═O)—R^{2a}, where R^{2a} is C₁-C₄-alkyl.

E.8. The method according to embodiment E.7, where the acylating agent used in step (i) or (iii) is a carboxylic acid anhydride R^{2a}—C(═O)—O—C(═O)—R^{2a}, where R^{2a} is methyl (the acylating agent thus being acetic anhydride).

E.9. The method according to any of the preceding embodiments, where the molar ratio of the compound of the formula II.a and the acylating agent is 20:1 to 1:5 if R¹ is —C(═O)R², and is 10:1 to 1:5 if R¹ is hydrogen.

E. 10. The method according to embodiment E.9, where the molar ratio of the compound of the formula II.a and the acylating agent is 5:1 to 1:5 if R¹ is —C(═O)R², and is 1:1 to 1:5 if R¹ is hydrogen.

E.11. The method according to embodiment E.10, where the molar ratio of the compound of the formula II.a and the acylating agent is 3:1 to 1:2 if R¹ is —C(═O)R², and is 1:1.1 to 1:3 if R¹ is hydrogen.

E. 12. The method according to embodiment E.11, where the molar ratio of the compound of the formula II.a and the acylating agent is 3:1 to 1:1 if R¹ is —C(═O)R², and is 1:1.5 to 1:3 if R¹ is hydrogen.

E.13. The method according to any of the preceding embodiments, where the photosensitizer is selected from the group consisting of fluorescein, eosin, rose bengal, erythrosine, tetraphenylporphyrin, cobalt-tetraphenylporphyrin, zinc-tetraphenylporphyrin, hematoporphyrin, rhodamine B, basacryl brilliant red, methyl violet, methylene blue, fullerene C₆₀, fullerene C₇₀, graphene, carbon nanotubes, Ru(bpy)₃²⁺ salts, Ru(phen)₃²⁺ salts, cercosporin, hypocrellin-A and mixtures thereof.

E.14. The method according to embodiment E.13, where the photosensitizer is selected from the group consisting of tetraphenylporphyrin, cobalt-tetraphenylporphyrin, zinc-tetraphenylporphyrin, methylene blue, Ru(bpy)₃²⁺ salts and Ru(phen)₃²⁺ salts.

E.15. The method according to embodiment E.14, where the photosensitizer is selected from the group consisting of from tetraphenylporphyrin, zinc-tetraphenylporphyrin and Ru(bpy)₃²⁺ salts.

E.16. The method according to any of the preceding embodiments, where in step (ii) further photosensitizer is added if this is depleted during irradiation.

E. 17. The method according to any of the preceding embodiments, where the photosensitizer is used in an overall amount of from 0.00001 to 1 mol-%, relative to 1 mol of the compound of the formula II.a.

E. 18. The method according to embodiment E.17, where the photosensitizer is used in an overall amount of from 0.0001 to 0.5 mol-%, relative to 1 mol of the compound of the formula II.a.

E.19. The method according to any of embodiments E.1 to E. 16, where the photosensitizer is used in an overall amount of from 0.000005 to 0.01 mol per mol of the compound of the formula II.a.

E.20. The method according to embodiment E.19, where the photosensitizer is used in an overall amount of from 0.00001 to 0.005 mol per mol of the compound of the formula II.a.

E.21. The method according to embodiment E.20, where the photosensitizer is used in an overall amount of from 0.0001 to 0.005 mol per mol of the compound of the formula II.a.

E.22. The method according to any of the preceding embodiments, where in step (ii) the reaction mixture is irradiated with light in the wavelength range of from 350 to 800 nm.

E.23. The method according to embodiment E.22, where in step (ii) the reaction mixture is irradiated with light in the wavelength range of from 350 to 680 nm.

E.24. The method according to embodiment E.23, where in step (ii) the reaction mixture is irradiated with light in the wavelength range of from 400 to 650 nm.

E.25. The method according to embodiment E.24, where in step (ii) the reaction mixture is irradiated with light in the wavelength range of from 400 to 580 nm.

E.26. The method according to embodiment E.25, where in step (ii) the reaction mixture is irradiated with light in the wavelength range of from 400 to 500 nm.

E.27. The method according to any of embodiments E. 14 to E.26, where the photosensitizer is tetraphenylporphyrin, cobalt-tetraphenylporphyrin or zinc-tetraphenylporphyrin and in step (ii) the reaction mixture is irradiated with light in the wavelength range of from 400 to 430 nm, preferably 400 to 420 nm, e.g. 400 to 410 nm; or the photosensitizer is methylene blue and in step (ii) the reaction mixture is irradiated with light in the wavelength range of from 600 to 620 nm, or the photosensitizer is a Ru(bpy)₃²⁺ salt or a Ru(phen)₃²⁺ salt and in step (ii) the reaction mixture is irradiated with light in the wavelength range of from 450 to 480 nm, preferably from 460 to 475 nm.

E.28. The method according to any of the preceding embodiments, where in step (ii) the reaction mixture is irradiated with monochromatic light.

E.29. The method according to embodiment E.28, where irradiation in step (ii) is carried out using a monochromatic light source, preferably an electroluminescent lighting device emitting monochromatic light, where at least 90% of the light emitted by said monochromatic light source is in the wavelength range of from 350 to 800 nm.

E.30. The method according to embodiment E.29, where irradiation in step (ii) is carried out using a monochromatic light source, preferably an electroluminescent lighting device emitting monochromatic light, where at least 90% of the light emitted by said monochromatic light source is in the wavelength range of from 350 to 680 nm.

E.31. The method according to embodiment E.30, where irradiation in step (ii) is carried out using a monochromatic light source, preferably an electroluminescent lighting device emitting monochromatic light, where at least 90% of the light emitted by said monochromatic light source is in the wavelength range of from 400 to 650 nm.

E.32. The method according to embodiment E.31, where irradiation in step (ii) is carried out using a monochromatic light source, preferably an electroluminescent lighting device emitting monochromatic light, where at least 90% of the light emitted by said monochromatic light source is in the wavelength range of from 400 to 580 nm.

E.33. The method according to embodiment E.32, where irradiation in step (ii) is carried out using a monochromatic light source, preferably an electroluminescent lighting device emitting monochromatic light, where at least 90% of the light emitted by said monochromatic light source is in the wavelength range of from 400 to 500 nm.

E.34. The method according to any of embodiments E.28 to E.33, where irradiation in step (ii) is carried out using an electroluminescent lighting device emitting monochromatic light, where the electroluminescent lighting device consists of at least one LED.

E.35. The method according to any of the preceding embodiments, where the oxygen-containing gas used in step (ii) is selected from the group consisting of oxygen, air and mixtures of oxygen and nitrogen containing oxygen in a range of from 1 to 99% by weight, relative to the total weight of the mixture (i.e. of oxygen and nitrogen).

E.36. The method according to embodiment E.35, where the oxygen-containing gas used in step (ii) is oxygen, air or a mixtures of oxygen and nitrogen containing oxygen in a range of from 20 to 99% by weight, relative to the total weight of the mixture.

E.37. The method according to embodiment E.36, where the oxygen-containing gas used in step (ii) is oxygen.

E.38. The method according to any of the preceding embodiments, where step (ii) is carried out neat.

E.39. The method according to any of embodiments E.1 to E.37, where step (ii) is carried out in the presence of a chlorinated C₁-C₂-alkane, where the molar ratio of the compound (II.a) provided in step (i) to the chlorinated C₁-C₂-alkane is of from 30:1 to 1:1.5, preferably from 10:1 to 1:1.5.

E.40. The method according to embodiment E.39, where the molar ratio of the compound (II.a) provided in step (i) to the chlorinated C₁-C₂-alkane is of from 5:1 to 1:1.

E.41. The method according to any of embodiments E.39 and E.40, where the chlorinated C₁-C₂-alkane is trichloromethane or tetrachloromethane.

E.42. The method according to any of the preceding embodiments, where in case that in step (i) an acylating agent has been provided, and the acylating agent is a carboxylic halide R^{2a}—C(═O)—X or a carboxylic acid anhydride R^{2a}—C(═O)—O—C(═O)R^{2a}, step (ii) is carried out in the presence of a base, and in case that in step (i) no acylating agent has been provided, in step (iii) also a base is added to the reaction mixture obtained in step (ii) if the acylating agent added in step (iii) is a carboxylic halide R^{2a}—C(═O)—X or a carboxylic acid anhydride R^{2a}—C(═O)—O—C(═O)R^{2a}.

E.43. The method according to embodiment E.42, where the base is an organic base.

E.44. The method according to embodiment E.43, where the base is selected from tertiary amines, basic 3- to 10-membered saturated, partially unsaturated or aromatic monocyclic or bicyclic heterocyclic rings containing 1, 2, 3 or 4 nitrogen atom as ring members, guanidines and amidines.

E.45. The method according to embodiment E.44, where the base is selected from the group consisting of
tertiary amines of the formula N(R³)₃, where each R³ is independently C₁-C₄-alkyl;
3- to 8-membered saturated monocyclic or bridged heterocyclic rings containing 1 or 2 nitrogen atoms or 1 nitrogen atom and one oxygen atom as ring members; in particular compounds of the formula NR³¹(R³²)₂, where R³¹ is hydrogen or C₁-C₄-alkyl and the two R³² form together a bridging group —(CH₂)ₙ—, where n is 2, 3, 4 or 5, or form together a bridging group —(CH₂)₂—N(R³³)—(CH₂)₂— or —(CH₂)₂—O—(CH₂)₂—, where R³³ is hydrogen or C₁-C₄-alkyl; or DABCO;
5- to 10-membered monocyclic or bicyclic heteroaromatic rings containing 1 or 2 nitrogen ring atoms as ring members; in particular selected from the group consisting of imidazole, pyridine, pyrazine, pyridazine, pyrimidine, quinolone and isoquinoline; where the monocyclic or bicyclic heteroaromatic rings are unsubstituted or carry 1, 2 or 3 C₁-C₄-alkyl substituents;
guanidines of the formula (R³)₂N—C(═NR³)—N(R³)₂, where each R³ is independently C₁-C₄-alkyl or two R³ bound to the same nitrogen atom or on different nitrogen atoms form together a bridging group —(CH₂)ₙ—, where n is 2, 3, 4 or 5; and
the bicyclic amidines DBN or DBU.

E.46. The method according to embodiment E.45, where in case that in step (i) an acylating agent has been provided, step (ii) is carried out in the presence of a base which is selected from 5- to 10-membered monocyclic or bicyclic heteroaromatic rings containing 1 or 2 nitrogen ring atoms as ring members.

E.47. The method according to embodiment E.6 where the base is selected from the group consisting of imidazole, pyridine, pyrazine, pyridazine, pyrimidine, quinolone and isoquinoline; where the monocyclic or bicyclic heteroaromatic rings are unsubstituted or carry 1, 2 or 3 C₁-C₄-alkyl substituents; and is in particular pyridine which is unsubstituted or carries 1, 2 or 3 C₁-C₄-alkyl substituents.

E.48. The method according to embodiment E.47, where the base is selected from pyridine, the picolines, the lutidines, the collidines and mixtures thereof.

E.49. The method according to embodiment E.48, where the base is pyridine.

E.50. The method according to any of embodiments E.42 to E.49, where the molar ratio of the compound of the formula II.a and the base is from 5:1 to 1:5.

E.51. The method according to embodiment E.50, where the molar ratio of the compound of the formula II.a and the base is from 3:1 to 1:3.

E.52. The method according to embodiment E.51, where the molar ratio of the compound of the formula II.a and the base is from 2:1 to 1:2.

E.53. The method according to embodiment E.52, where the molar ratio of the compound of the formula II.a and the base is from 1.5:1 to 1:2, e.g. 1.5:1 to 1:1.5.

E.54. The method according to any of the preceding embodiments, where in case that in step (i) an acylating agent has been provided, step (ii) is carried out in the presence of an acylation catalyst, and in case that in step (i) no acylating agent has been provided, in step (iii) also an acylation catalyst is added to the reaction mixture obtained in step (ii).

E.55. The method according to embodiment E.54, where the acylation catalyst is selected from the group consisting of 4-dimethylaminopyridine (DMAP), 4-pyrrolidinopyridine (PPY), 1,6-dibenzyl-2,3,5,6-tetrahydro-1H,4H-1,3a,6,8-tetraazaphenalene (Super DMAP) and acylation catalysts of the general formulae

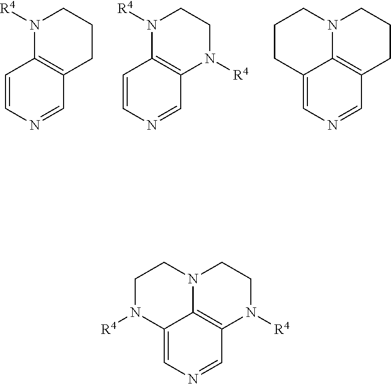

where each R⁴ is independently methyl or ethyl.

E.56. The method according to embodiment E.55, where the acylation catalyst is 4-dimethylaminopyridine (DMAP).

E.57. The method according to any of embodiments E.52 to E.56, where the molar ratio of the compound of the formula II.a and the acylation catalyst is from 200:1 to 1:1.

E.58. The method according to embodiment E.57, where the molar ratio of the compound of the formula II.a and the acylation catalyst is from 200:1 to 5:1.

E.59. The method according to embodiment E.58, where the molar ratio of the compound of the formula II.a and the acylation catalyst is from 150:1 to 10:1.

E.60. The method according to any of the preceding embodiments, where step (ii) is carried out at a temperature of from −20 to 150° C.

E.61. The method according to embodiment E.60, where step (ii) is carried out at a temperature of from 0 to 70° C., e.g. 0 to 60° C. or 5 to 50° C.

E.62. The method according to any of the preceding embodiments, where step (ii) is carried out at a pressure of from atmospheric pressure to 100 bar (10 MPa).

E.63. The method according to embodiment E.62, where step (ii) is carried out at from >1 to 10 bar (>0.1 to 1 MPa).

E.64. The method according to embodiment E.63, where step (ii) is carried out at from 1.5 to 8 bar (0.15 to 0.8 MPa).

E.65. The method according to embodiment E.64, where step (ii) is carried out at from 5 to 20 bar (0.5 to 2 MPa).

E.66. The method according to embodiment E.65, where step (ii) is carried out at from 10 to 15 bar (1 to 1.5 MPa).

E.67. The method according to embodiment E.66, where step (ii) is carried out at atmospheric pressure.

E.68. The method according to any of the preceding embodiments, where in step (ii) either the complete reaction mixture or only a distinct portion of the reaction mixture is irradiated.

E.69. The method according to any of the preceding embodiments, where step (ii) is carried out in a side-loop photoreactor, a continuous flow-photoreactor or a submersible photoreactor.

E.70. The method according to any of the preceding embodiments, where in step (i) an acylating agent, a base and optionally an acylation catalyst is provided (and step (iii) is not carried out).

E.71. The method according to embodiment E.70, where in step (i) an acylating agent, an acylation catalyst and a base is provided (and step (iii) is not carried out).

E.72. The use of the compound of the formula I.a or I.b different from (E)-4-acetoxy-2-methylbut-2-enal or of a stereoisomer of the compound I.a or I.b different from (E)-4-acetoxy-2-methylbut-2-enal or of a mixture of different stereoisomers of the compound I.a and/or I.b or of a mixture of different compounds I.a and/or I.b as defined in any of embodiments E.1 to E.5, as intermediates in the synthesis of retinol, stereoisomers thereof, derivatives thereof (preferably esters thereof) or stereoisomers of derivatives thereof (preferably stereoisomers of esters thereof).

E.73. A hydroperoxide compound of the formula III.a, III.b or III.c or a stereoisomer of the compound of the formula III.a, III.b or III.c or a mixture of different stereoisomers of the compound III.a, III.b and/or III.c or a mixture of different compounds III.a, III.b and/or III.c

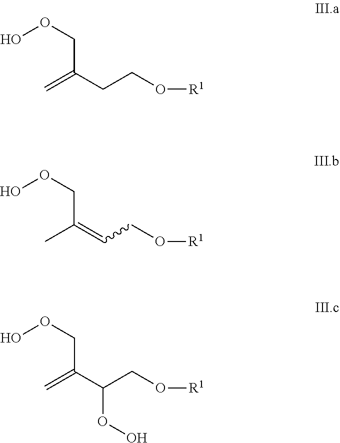

where
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl.

E.74. The hydroperoxide compound according to embodiment E.73, where R² is C₁-C₄-alkyl.

E.75. The hydroperoxide compound according to embodiment E.74, where R² is methyl.

E.76. The hydroperoxide compound according to any of embodiments E.73 to E.75, which is a compound of the formula III.a or III.b or a stereoisomer of the compound of the formula III.a or III.b or a mixture of different stereoisomers of the compound III.a and/or III.b or a mixture of different compounds III.a and/or III.b.

E.77. The hydroperoxide compound according to any of embodiments E.73 to E.75, which is a compound of the formula III.a or III.c or a stereoisomer of the compound of the formula III.a or III.c or a mixture of different stereoisomers of the compound III.a and/or III.c or a mixture of different compounds III.a and/or III.c.

E.78. The hydroperoxide compound according to any of embodiments E.73 to E.75, which is a compound of the formula III.a or a stereoisomer of the compound of the formula III.a or a mixture of different stereoisomers of the compound III.a or a mixture of different compounds III.a.

E.79. The use of the hydroperoxide compound of the formula III.a, III.b or III.c or of a stereoisomer of the compound of the formula III.a, III.b or III.c or of a mixture of different stereoisomers of the compound III.a, and/or III.b and/or III.c or of a mixture of different compounds III.a, III.b and/or III.c as defined in any of embodiments E.73 to E.75, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined in any of embodiments E.1 to E.5, or as intermediates in the synthesis of retinol, stereoisomers thereof, derivatives thereof (preferably esters thereof) or stereoisomers of derivatives thereof (preferably stereoisomers of esters thereof).

E.80. The use according to embodiment E.79, of the hydroperoxide compound of the formula III.a or III.b or of a stereoisomer of the compound of the formula III.a or III.b or of a mixture of different stereoisomers of the compound III.a and/or III.b or of a mixture of different compounds III.a and/or III.b.

E.81. The use according to embodiment E.79, of the hydroperoxide compound of the formula III.a or a stereoisomer of the compound of the formula III.a or a mixture of different stereoisomers of the compound III.a or a mixture of different compounds III.a.

Without wishing to be bound by theory, it is assumed that in the method of the invention the conversion of compounds of the formula II.a into compounds I.a and/or I.b proceeds via a Schenck ene reaction (=an “ene” reaction in which singlet oxygen is the enophile) and simultaneous (if step (iii) is not carried out) or subsequent (if step (iii) is carried out) Kornblum-DeLaMare rearrangement (which is actually an elimination reaction; here a dehydroacylation).

In the method of the invention the conversion of the compounds of the formula II.a into compounds I.a and/or I.b can be carried out in one step, e.g. as a one-pot-reaction, if the acylating agent is provided in step (i). Step (iii) is of course not carried out in this case. Without wishing to be bound by theory, it is assumed that in the course of step (ii) the compound of the formula II.a is converted into the allylic hydroperoxide which is acylated in situ by the acylating agent. The acylated hydroperoxide is then dehydrated in situ, to be more precise dehydroacylated, to afford compounds I.a and/or I.b. Compounds I.b are assumed to be the result of a double bond isomerization which can occur during the formation of the hydroperoxides and can lead to hydroperoxide intermediates III.b as depicted above or below.

Alternatively, in the method of the invention the conversion of the compounds of the formula II.a into compounds I.a and/or I.b can be carried out in two steps if the acylating agent is not provided in step (i). In this case, step (iii) is mandatory. In the course of step (ii), hydroperoxides are formed which upon addition of the acylating agent in step (iii) are acylated and react further to compounds I.a and/or I.b as explained above.

Given the safety risks associated with the formation and longer storage of hydroperoxides, and also for the simpler handling, the first alternative, i.e. converting the compounds of the formula II.a into compounds I.a and/or I.b in one step by providing in step (i) a reaction mixture comprising the acylating agent, is preferred.

Thus, in a preferred embodiment, the invention relates to a method for preparing a compound of the formula I.a or of the formula I.b or a mixture thereof or a stereoisomer of the compound I.b or a mixture of different stereoisomers of the compound I.b or a mixture of different compounds I.a and/or I.b

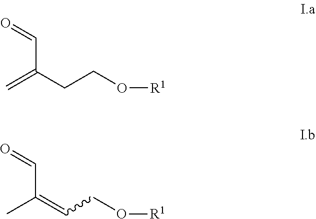

wherein
R¹ is hydrogen or —C(═O)R²; and
R² is C₁-C₂₀-alkyl;
which method comprises
(i) providing a reaction mixture comprising a compound of the formula II.a

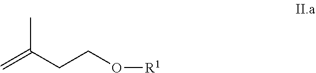

wherein R¹ is as defined above;
a photosensitizer and an acylating agent;
(ii) passing an oxygen-containing gas through the reaction mixture provided in step (i) and simultaneously irradiating the reaction mixture with light;
(iv.1) if desired, after completion of the reaction (to the desired degree), isolating the one or more compounds (I.a) or (I.b) obtained in step (ii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (ii); and
(v.2) if desired, isolating the one or more compounds (I.a) or (I.b) obtained in step (iv.2).

In compounds I.a, I.b and II.a, R² is preferably C₁-C₄-alkyl or n-C₁₅-alkyl, more preferably C₁-C₄-alkyl, even more preferably C₁-C₂-alkyl and in particular methyl.

R¹ is preferably-C(═O)R², especially in compounds II.a.

The acylating agent used in step (i) or (iii) can principally be any substance which can acylate the hydroperoxides formed in the reaction of compounds II.a with oxygen. Typical acylating agents are carboxylic acids, carboxylic active esters, carboxylic halides, carboxylic anhydrides and ketenes. Preferably, the acylating agent is selected from the group consisting of carboxylic halides R^{2a}—C(═O)—X, carboxylic acid anhydrides R^{2a}—C(═O)—O—C(═O)—R^{2a}, and ketenes R^{2a}—C(H)═C═O, where R^{2a} has independently one of the general or preferred meanings given above for R² and X is Cl, Br or I. More preferably, the acylating agent used in step (i) or (iii) is a carboxylic acid anhydride R^{2a}—C(═O)—O—C(═O)—R^{2a}, even more preferably a carboxylic acid anhydride R^{2a}—C(═O)—O—C(═O)—R^{2a} where R^{2a} is C₁-C₄-alkyl, and is in particularly a carboxylic acid anhydride R^{2a}—C(═O)—O—C(═O)—R^{2a} where R^{2a} is methyl, the carboxylic acid anhydride being thus acetic anhydride.

The acylating agent is provided in such amounts that at least a part of the hydroperoxide group formed is acylated. If in compounds II.a R¹ is hydrogen, acylation of the alcoholic group O—R¹═OH competes with the acylation of the hydroperoxide group. It is therefore expedient in this case to use the acylating agent in sufficiently high amounts to allow acylation of at least a part of the hydroperoxide group.

Preferably, the acylating agent is provided in such amounts that the molar ratio of the compound of the formula II.a and the acylating agent is 20:1 to 1:5 if R¹ is —C(═O)R², and is 10:1 to 1:5 if R¹ is hydrogen. More preferably, the molar ratio of the compound of the formula II.a and the acylating agent is 5:1 to 1:5 if R¹ is —C(═O)R², and is 1:1 to 1:5 if R¹ is hydrogen. Even more preferably, the molar ratio of the compound of the formula II.a and the acylating agent is 3:1 to 1:2 if R¹ is —C(═O)R², and is 1:1.1 to 1:3 if R¹ is hydrogen. In particular, the molar ratio of the compound of the formula II.a and the acylating agent is 3:1 to 1:1 if R¹ is —C(═O)R², and is 1:1.5 to 1:3 if R¹ is hydrogen.

The above ratios relate to the amount of compound II.a as provided in step (i) and take also account of the above- or below-described preferred embodiment according to which the reaction is carried out thusly that only a part of compound II.a is converted.

As explained above, the photosensitizer used in the method of the invention is a compound which, when subjected to irradiation (generally to electromagnetic radiation in the UV, visible or in the near IR region) can convert triplet oxygen to singlet oxygen: Upon irradiation, the sensitizer forms the corresponding excited singlet state. Intersystem crossing affords the excited triplet state of the sensitizer, thus transferring energy to triplet oxygen to form singlet oxygen. Singlet oxygen is the species which oxidizes the compounds II.a to the respective hydroperoxides (or double bond isomers thereof).

Preferably, the photosensitizer can convert triplet oxygen to singlet oxygen when subjected to electromagnetic radiation in the near UV, in the visible or in the near IR region, more preferably in the visible or in the near IR region, in particular in the visible region.

Preferably, the photosensitizer is selected from the group consisting of fluorescein, eosin, rose Bengal (RB), erythrosine, tetraphenylporphyrin (to be more precise 5,10,15,20-tetraphenyl-21H,23H-porphine; TPP; 2HTPP), cobalt-tetraphenylporphyrin (Co-TPP; i.e. the cobalt complex of TPP with Co(II)), zinc-tetraphenylporphyrin (Zn-TPP; i.e. the zinc complex of TPP with Zn(II)), hematoporphyrin, rhodamine B, basacryl brilliant red, methyl violet, methylene blue, fullerene C₆₀, fullerene C₇₀, graphene, carbon nanotubes, Ru(bpy)₃²⁺ salts (bpy=2,2′-bipyridine) (e.g. tris(2,2′-bipyridine) ruthenium (II) hexafluorophosphate, tris(2,2′-bipyridine)ruthenium (II) chloride, often as hexahydrate) Ru(phen)₃²⁺ salts (phen=1,10-phenanthroline) (e.g. dichlorotris (1,10-phenanthroline)ruthenium (II) chloride), cercosporin, hypocrellin-A and mixtures thereof. More preferably, the photosensitizer is selected from the group consisting of tetraphenylporphyrin, cobalt-tetraphenylporphyrin, zinc-tetraphenylporphyrin, methylene blue, Ru(bpy)₃²⁺ salts (in particular tris(2,2′-bipyridine)ruthenium (II) hexafluorophosphate, tris(2,2′-bipyridine)ruthenium (II) chloride or its hexahydrate) and Ru(phen)₃²⁺ salts (in particular dichlorotris(1,10-phenanthroline)ruthenium (II) chloride), and in particular from tetraphenylporphyrin, zinc-tetraphenylporphyrin and Ru(bpy)₃²⁺ salts (in particular tris(2,2′-bipyridine)ruthenium (II) hexafluorophosphate, tris(2,2′-bipyridine)ruthenium (II) chloride or its hexahydrate).

The photosensitizer can be provided in step (i) in very low amounts. However, during irradiation, a part of the photosensitizer may degrade, leading to decreasing generation of singlet oxygen and thus slowing down the conversion of the compounds II.a to the corresponding hydroperoxides and eventually to the desired compounds I.a and/or I.b. Thus, either higher amounts of the photosensitizer are provided in step (i) or in the course of step (ii) further photosensitizer is added if this is depleted during irradiation. The degree of depletion/degradation of the photosensitizer can be monitored in the course of step (ii), e.g. by UV/Vis spectroscopy, which can also be carried out in line.

The photosensitizer is preferably used in an overall amount of from 0.00001 to 1 mol-%, relative to 1 mol of the compound of the formula II.a. Overall amount means the total amount of photosensitizer provided in step (i) and added in the course of step (ii), if applicable. More preferably, the photosensitizer is used in an overall amount of from 0.0001 to 0.5 mol-%, relative to 1 mol of the compound of the formula II.a.

Alternatively, the photosensitizer is preferably used in an overall amount of from 0.0000001 to 0.01 mol, more preferably from 0.000001 to 0.01 mol or from 0.000001 to 0.005 mol, even more preferably from 0.000005 to 0.01 mol, particularly preferably from 0.00001 to 0.005 mol, specifically from 0.0001 to 0.005 mol, per 1 mol of the compound of the formula II.a.

Typical photosensitizers are dyes and are thus excitable with electromagnetic radiation in the near UV, visible or near infrared (near IR; NIR) electromagnetic spectrum. Thus, preferably in step (ii) the reaction mixture is irradiated with light in the near UV, visible or near IR range. More preferably, in step (ii) the reaction mixture is irradiated with light in the visible or near IR range, and in particular in the visible range.

Preferably, in step (ii) the reaction mixture is irradiated with light in the wavelength range of from 350 to 800 nm, more preferably from 350 to 680 nm, even more preferably in the wavelength range of from 400 to 650 nm, even more preferably from 400 to 580 nm, and in particular from 400 to 500 nm. The optimum wavelength range depends i.a. on the photosensitizer used and can for example be determined by short tests, if not anyway known to the skilled person, or can be selected by means of UV spectroscopy.

For instance, if the photosensitizer is tetraphenylporphyrin, cobalt-tetraphenylporphyrin or zinc-tetraphenylporphyrin, in step (ii) the reaction mixture can for example be irradiated with light in the wavelength range of from 400 to 430 nm, preferably 400 to 420 nm, e.g. 400 to 410 nm; if the photosensitizer is methylene blue, in step (ii) the reaction mixture can for example be irradiated with light in the wavelength range of from 600 to 620 nm, and if the photosensitizer is a Ru(bpy)₃²⁺ salt or a Ru(phen)₃²⁺ salt, in step (ii) the reaction mixture can for example be irradiated with light in the wavelength range of from 450 to 480 nm, preferably from 460 to 475 nm.

In step (ii) the reaction mixture is preferably irradiated with monochromatic light.

In theory, monochromatic light is light with a single constant frequency/light of a single vacuum wavelength range. In practice, however, no radiation can be totally monochromatic. Thus, in practice, “monochromatic” light-even from lasers or spectral lines—always consists of components with a range of frequencies of non-zero width. In terms of the present invention, monochromatic light is understood as light produced by state-of-the-art sources of monochromatic light, such as monochromators, optical filters, Hg vapour lamps (high, middle or low pressure lamps), generally in combination with an optical filter, doped Hg vapour lamps, if necessary in combination with an optical filter, Na vapour lamps (high or low pressure lamps), lasers, or, in particular, monochromatic LEDs.

Preferably, irradiation in step (ii) is carried out using a monochromatic light source, preferably an electroluminescent lighting device emitting monochromatic light, where at least 90% of the light emitted by said monochromatic light source is in the wavelength range of from 350 to 800 nm, preferably from 350 to 680 nm, more preferably in the wavelength range of from 400 to 650 nm, even more preferably from 400 to 580 nm, in particular from 400 to 500 nm.

Preferably, irradiation in step (ii) is carried out using an electroluminescent lighting device emitting monochromatic light, where the electroluminescent lighting device consists of at least one LED.

The oxygen-containing gas used in step (ii) is preferably selected from the group consisting of oxygen, air and mixtures of oxygen and nitrogen containing oxygen in a range of from 1 to 99% by weight, relative to the total weight of the mixture. More preferably, the oxygen-containing gas used in step (ii) is selected from the group consisting of oxygen, air and mixtures of oxygen and nitrogen containing oxygen in a range of from 20 to 99% by weight, and is in particular oxygen.

“Passing an oxygen-containing gas through the reaction mixture provided in step (i)” is not limited to bubbling an oxygen-containing gas through said mixture, thus letting a substantial part of the oxygen-containing gas escape, but also encompasses inserting into and keeping an oxygen-containing gas in the reaction mixture, e.g. by using a closed, generally pressurized reaction vessel.

In case that in step (i) an acylating agent has been provided, step (ii) is preferably carried out in the presence of a base, especially if the acylating agent is a carboxylic acid halide or a carboxylic acid anhydride. The base in this case serves especially for neutralizing the acid formed (HX or R^{2a}COOH). The base is preferably an organic base. In this case, step (i) preferably comprises providing a reaction mixture comprising a compound of the formula II.a, a photosensitizer, an acylating agent and a base, preferably an organic base.

In case that in step (i) no acylating agent has been provided, in step (iii) also a base, preferably of an organic base, is added to the reaction mixture obtained in step (ii), especially if the acylating agent is a carboxylic acid halide or a carboxylic acid anhydride. In this case, step (iii) preferably comprises adding an acylating agent and a base, preferably an organic base, to the reaction mixture obtained in step (ii).

The organic base preferably added in steps (i) or (iii) is preferably selected from tertiary amines, basic 3- to 10-membered saturated, partially unsaturated or aromatic monocyclic or bicyclic heterocyclic rings containing 1, 2, 3 or 4 nitrogen atom as ring members, guanidines and amidines.

More preferably, the base is selected from the group consisting of:
tertiary amines of the formula N(R³)₃, where each R³ is independently C₁-C₄-alkyl;
3- to 8-membered saturated monocyclic or bridged heterocyclic rings containing 1 or 2 nitrogen atoms or 1 nitrogen atom and one oxygen atom as ring members; in particular compounds of the formula NR³¹(R³²)₂, where R³¹ is hydrogen or C₁-C₄-alkyl and the two R³² form together a bridging group —(CH₂)ₙ—, where n is 2, 3, 4 or 5, or form together a bridging group —(CH₂)₂—N(R³³)—(CH₂)₂— or —(CH₂)₂—O—(CH₂)₂—, where R³³ is hydrogen or C₁-C₄-alkyl; or DABCO;
5- to 10-membered monocyclic or bicyclic heteroaromatic rings containing 1 or 2 nitrogen ring atoms as ring members; in particular selected from the group consisting of imidazole, pyridine, pyrazine, pyridazine, pyrimidine, quinolone and isoquinoline; where the monocyclic or bicyclic heteroaromatic rings are unsubstituted or carry 1, 2 or 3 C₁-C₄-alkyl substituents;
guanidines of the formula (R³)₂N—C(═NR³)—N(R³)₂, where each R³ is independently C₁-C₄-alkyl or two R³ bound to the same nitrogen atom or on different nitrogen atoms form together a bridging group —(CH₂)ₙ—, where n is 2, 3, 4 or 5; and
the bicyclic amidines DBN or DBU.

Examples for tertiary amines of the formula N(R³)₃, where each R³ is independently C₁-C₄-alkyl, are trimethylamine, triethylamine, tri-n-propylamine, tri-n-butylamine, diisopropylethyl amine (Hünig's base) and the like.

Examples for 3- to 8-membered saturated monocyclic or bridged heterocyclic rings containing 1 or 2 nitrogen atoms or 1 nitrogen atom and one oxygen atom as ring members are piperidine, 1-methylpiperidine, 1-ethylpiperidine, piperazine, morpholine, 1,4-diazabicyclo[2.2.2]octane (DABCO) and the like.

Examples for 5- to 10-membered monocyclic or bicyclic heteroaromatic rings containing 1 or 2 nitrogen ring atoms and unsubstituted or substituted 1, 2 or 3 C₁-C₄-alkyl substituents as ring members are imidazole, pyridine, the picolines, the lutidines, pyrazine, pyridazine, pyrimidine, quinolone and isoquinoline.

Where step (ii) is to be carried out in the presence of a base, the latter is advantageously selected from 5- to 10-membered monocyclic or bicyclic heteroaromatic rings containing 1 or 2 nitrogen ring atoms as ring members (thus, expediently, the reaction mixture provided in step (i) advantageously comprises a base selected from 5- to 10-membered monocyclic or bicyclic heteroaromatic rings containing 1 or 2 nitrogen ring atoms as ring members if step (ii) is to be carried out in the presence of a base), since some of the other bases, especially tertiary amines of the formula N(R³)₃, may interfere negatively in the photooxidation reaction. More preferably, the base is selected from the group consisting of imidazole, pyridine, pyrazine, pyridazine, pyrimidine, quinolone and isoquinoline; where the monocyclic or bicyclic heteroaromatic rings are unsubstituted or carry 1, 2 or 3 C₁-C₄-alkyl substituents; and is particularly preferably pyridine which is unsubstituted or carries 1, 2 or 3 C₁-C₄-alkyl substituents. In particular, the base is selected from pyridine, the picolines (i.e. 2-methylpyridine, 3-methylpyridine, 4-methylpyridine or mixtures thereof) the lutidines (i.e. 2,3-dimethylpyridine, 2,4-dimethylpyridine, 2,5-dimethylpyridine, 2,6-dimethylpyridine, 3,4-dimethylpyridine, 3,5-dimethylpyridine or mixtures thereof), the collidines (i.e. 2,3,4-trimethylpyridine, 2,3,5-trimethylpyridine, 2,3,6-trimethylpyridine, 2,4,5-trimethylpyridine, 2,4,6-trimethylpyridine, 3,4,5-trimethylpyridine and mixtures thereof) and mixtures thereof, and is even more particularly pyridine.

If in step (i) no acylating agent has been provided and step (iii) is carried out, the base preferably added in step (iii) can be any of the above-mentioned organic bases, since no negative interference in the photooxidation reaction is possible in this set-up. However, preference is nevertheless given to the same bases as those mentioned above for the base to be added in step (i).

The base is preferably used in such amounts that the molar ratio of the compound of the formula II.a and the base is from 5:1 to 1:5, more preferably from 3:1 to 1:3, even more preferably from 2:1 to 1:2 and in particular from 1.5:1 to 1:2 or from 1.5:1 to 1:1.5.

In case that in step (i) an acylating agent has been provided, step (ii) is preferably carried out in the presence of an acylation catalyst. In this case, step (i) preferably comprises providing a reaction mixture comprising a compound of the formula II.a, a photosensitizer, an acylating agent, an acylation catalyst and optionally a base, preferably an organic base (different from the acylation catalyst; see below). More preferably, step (i) comprises providing a reaction mixture comprising a compound of the formula II.a, a photosensitizer, an acylating agent, an acylation catalyst and a base, preferably an organic base (different from the acylation catalyst; see below).

In case that in step (i) no acylating agent has been provided, in step (iii) preferably also an acylation catalyst is added to the reaction mixture obtained in step (ii). In this case, step (iii) preferably comprises adding an acylating agent, an acylation catalyst and optionally a base, preferably an organic base (different from the acylation catalyst; see below), to the reaction mixture obtained in step (ii). More preferably, step (iii) comprises adding an acylating agent, an acylation catalyst and a base, preferably an organic base (different from the acylation catalyst; see below), to the reaction mixture obtained in step (ii).

Preferably, the acylation catalyst is selected from the group consisting of 4-dimethylaminopyridine (DMAP), 4-pyrrolidinopyridine (PPY), 1,6-dibenzyl-2,3,5,6-tetrahydro-1H,4H-1,3a,6,8-tetraazaphenalene (Super DMAP) and acylation catalysts of the general formulae

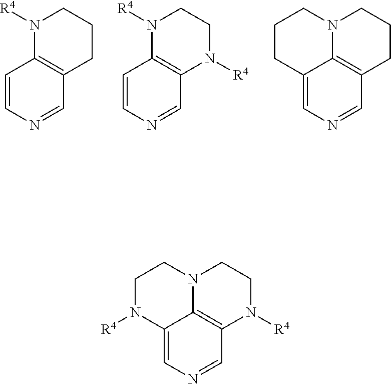

where each R⁴ is independently methyl or ethyl;

More preferably, the acylation catalyst is 4-dimethylaminopyridine (DMAP).

The acylation catalyst is preferably used in such amounts that the molar ratio of the compound of the formula II.a and the acylation catalyst is preferably from 200:1 to 1:1, more preferably from 200:1 to 5:1 and in particular from 150:1 to 10:1.

Preferably, step (ii) is carried out neat (i.e. in substance). “Neat” or “in substance” means that no additional solvent is present. The specification “additional” takes account of the fact that the starting compound II.a and also the intermediately formed hydroperoxides or the optional base can serve as solvent or dispersant for the photosensitizer, the acylating agent, if present, the optionally present acylation catalyst and the optionally present base (if the latter does not act itself as a solvent). To carry out step (ii) neat, the reaction mixture provided in step (i) is for example either prepared by mixing the starting materials (compounds II.a, photosensitizer, acylating agent if the reaction is to be carried out in one step, optionally acylation catalyst, optionally a base) in the absence of any additional solvent, or by mixing the starting materials in the presence of an additional solvent and then removing the same before step (ii) is carried out.

In an alternatively preferred embodiment, step (ii) is carried out in the presence of a chlorinated C₁-C₂-alkane. To this purpose, said chlorinated C₁-C₂-alkane is expediently provided in the reaction mixture of step (i). The molar ratio of the compound (II.a) provided in step (i) to the chlorinated C₁-C₂-alkane is of from 30:1 to 1:1.5, preferably from 10:1 to 1:1.5, more preferably from 5:1 to 1:1. The chlorinated C₁-C₂-alkane is preferably trichloromethane or tetrachloromethane.

More preferably, however, step (ii) is carried out neat.

In a preferred embodiment of the method of the invention, in step (i) the compound II.a, an acylating agent, a photosensitizer, a base and optionally an acylation catalyst is provided (and step (iii) is not carried out), and step (ii) is carried out neat. More preferably, in step (i) the compound II.a, a photosensitizer, an acylating agent, a base and an acylation catalyst is provided (and step (iii) is not carried out), and step (ii) is carried out neat. In a particular embodiment, in step (i) the compound II.a, a photosensitizer (in particular TPP, Zn-TPP or a Ru(bpy)₃²⁺ salt (e.g. tris(2,2′-bipyridine)ruthenium (II) hexafluorophosphate or tris(2,2′-bipyridine)ruthenium (II) chloride)), acetic anhydride, pyridine or pyridine carrying 1, 2 or 3 methyl groups and optionally DMAP is provided (and step (iii) is not carried out), and step (ii) is carried out neat. More particularly, in step (i) the compound II.a, a photosensitizer (in particular TPP, Zn-TPP or a Ru(bpy)₃²⁺ salt (e.g. tris(2,2′-bipyridine)ruthenium (II) hexafluorophosphate or tris(2,2′-bipyridine)ruthenium (II) chloride)), acetic anhydride, pyridine or pyridine carrying 1, 2 or 3 methyl groups and DMAP is provided (and step (iii) is not carried out), and step (ii) is carried out neat. Even more particularly, in step (i) the compound II.a, a photosensitizer (in particular TPP, Zn-TPP or a Ru(bpy)₃²⁺ salt (e.g. tris(2,2′-bipyridine)ruthenium (II) hexafluorophosphate or tris(2,2′-bipyridine)ruthenium (II) chloride)), acetic anhydride, pyridine and DMAP is provided (and step (iii) is not carried out), and step (ii) is carried out neat.

Step (ii) is preferably carried out at a temperature of from −20 to 150° C., more preferably from 0 to 70° C., e.g. 0 to 60° C. or 5 to 50° C.

Step (ii) is preferably carried out at a pressure of from atmospheric pressure to 100 bar (10 MPa). Atmospheric pressure means the local ambient pressure, and thus roughly 1013.25 hPa±200 hPa. In a more preferred embodiment, step (ii) is carried out at a pressure of from >1 to 10 bar (>0.1 to 1 mPa), in particular from 1.5 to 8 bar (0.15 to 0.8 MPa). In another more preferred embodiment, step (ii) is carried out at a pressure of from 5 to 20 bar (0.5 to 2 MPa) and in particular from 10 to 15 bar (1 to 1.5 MPa). In yet another more preferred embodiment, step (ii) is carried out at atmospheric pressure.

In step (ii) either the complete reaction mixture or only a distinct portion of the reaction mixture is irradiated. The latter occurs for example if only a portion of the reaction mixture (e.g. only 20 to 90% or 30 to 80% or 50 to 80% by weight of the reaction mixture) is passed by the irradiation source.

Step (ii) can be carried out in any reactor known in the art as suitable for photooxidations. Suitable reactors contain at least a means for introducing the oxygen-containing gas and a radiation source. Moreover, the reactor expediently contains a stirrer and means for cooling or heating.

Examples for suitable reactors are side-loop photoreactors, continuous flow-photoreactors or submersible photoreactors.

In a specific embodiment, step (ii) is carried out thus that a part of the starting compound II.a remains unreacted. This ensures that compound II.a can still serve as solvent for the other substances present in the reaction mixture. Preferably, at least 20%, more preferably at least 50% of the initially charged amounts of compound II.a remain unreacted in step (ii). After work-up, isolation and if desired purification, compound II.a can be reused in step (i). The degree of conversion of compounds II.a can be determined via usual means, such as periodical or continuous sample collection and analysis or in-line analysis of the composition of the reaction mixture, or by passing oxygen through the reaction mixture in predetermined substoichiometric amounts. Reaction in step (ii) is for example interrupted by ceasing the oxygen feed and/or by ceasing irradiation.

If in step (i) no acylating agent has been added and thus step (iii) is carried out, after completion of the reaction of step (ii) to the desired degree, the acylating agent, optionally an acylation catalyst and optionally a base are added to the reaction mixture obtained in step (ii). For safety reasons, it is expedient not to isolate the hydroperoxides formed in step (ii) before subjecting them to the acylation/dehydroacylation step (iii), but to use the reaction mixture directly in step (iii).

Step (iii) can be carried out neat (=in substance), or in the presence of a solvent. Generally, most common solvents can be used. Suitable solvents are for example aliphatic hydrocarbons, such as pentane, hexane or heptane; cycloaliphatic hydrocarbons, such as cyclohexane, cycloheptane or cyclooctane; aromatic hydrocarbons, such as benzene, toluene or the xylenes; halogenated aliphatic hydrocarbons, such as chlorinated C₁-C₂-alkanes, e.g. dichloromethane (methylene chloride), trichloromethane (chloroform), tetrachloromethane (carbon tetrachloride), 1,1-dichloroethane, 1,2-dichloroethane, 1,1,1-trichloroethane, 1,1,2-trichloroethane, 1,1,1,2-tetrachloroethane, 1,1,2,2-tetrachloroethane and pentachloroethane; or chloro-fluoro-C₁-C₂-alkanes, e.g. fluorotrichloromethane; aromatic hydrocarbons, such as chlorobenzene, dichlorobenzene or hexafluorobenzene; C₁-C₄-alkanols, such as methanol, ethanol, n-propanol, isopropanol and the butanols, C₂-C₃-alkanediols (glycols), such as ethylene glycol or propylene glycol; open-chained ethers, such as diethyl ether, di-n-propyl ether or methyl-tert-butyl ether; cyclic ethers, such as tetrahydrofuran or 1,4-dioxane; ketones, such as acetone or ethylmethylketone; C₁-C₃-alkyl esters of C₂-C₃-carboxylic acids, such as methyl acetate, ethyl acetate, propyl acetate, methyl propionate, ethyl propionate or propyl propionate; nitriles, such as acetonitrile, carboxamides, such as N, N,-dimethyl formamide, N,N,-diethyl formamide, N,N,-dimethyl acetamide or N,N,-diethyl acetamide; sulfoxides, such as dimethylsulfoxide; or CS₂. Among these, preference is given to chlorinated C₁-C₂-alkanes, chloro-fluoro-C₁-C₂-alkanes and CS₂, and more preference to chlorinated C₁-C₂-alkanes. Preferably, however, step (iii) is carried out neat. “Neat” or “in substance” means that no additional solvent is present. Regarding the specification “additional”, reference is made to the remarks made above in context with step (i).

In a preferred embodiment of the method of the invention, in step (i) the compound II.a, an acylating agent, a photosensitizer, a base and optionally an acylation catalyst is provided (and step (iii) is not carried out), and step (ii) is carried out neat. More preferably, in step (i) the compound II.a, a photosensitizer, an acylating agent, a base and an acylation catalyst is provided (and step (iii) is not carried out), and step (ii) is carried out neat. In a particular embodiment, in step (i) the compound II.a, a photosensitizer (in particular TPP, Zn-TPP or a Ru(bpy)₃²⁺ salt (e.g. tris(2,2′-bipyridine)ruthenium (II) hexafluorophosphate or tris(2,2′-bipyridine)ruthenium (II) chloride)), acetic anhydride, pyridine or pyridine carrying 1, 2 or 3 methyl groups and optionally DMAP is provided (and step (iii) is not carried out), and step (ii) is carried out neat. More particularly, in step (i) the compound II.a, a photosensitizer (in particular TPP, Zn-TPP or a Ru(bpy)₃²⁺ salt (e.g. tris(2,2′-bipyridine)ruthenium (II) hexafluorophosphate or tris(2,2′-bipyridine)ruthenium (II) chloride)), acetic anhydride, pyridine or pyridine carrying 1, 2 or 3 methyl groups and DMAP is provided (and step (iii) is not carried out), and step (ii) is carried out neat. Even more particularly, in step (i) the compound II.a, a photosensitizer (in particular TPP, Zn-TPP or a Ru(bpy)₃²⁺ salt (e.g. tris(2,2′-bipyridine)ruthenium (II) hexafluorophosphate or tris(2,2′-bipyridine)ruthenium (II) chloride)), acetic anhydride, pyridine and DMAP is provided (and step (iii) is not carried out), and step (ii) is carried out neat.

After completion of the reaction, the reaction mixture obtained in step (ii) (if step (iii) is not carried out) or step (iii) is generally worked up. “Completion” of the reaction in this context does not mandatorily mean maximum conversion of the starting material, but conversion to a desired degree. As explained above, especially if step (ii) is carried out neat, the starting compound II.a generally serves as solvent. In this case, it is expedient to stop the reaction distinctly before maximum conversion of II.a.

Work-up of the reaction mixture obtained in step (ii) (if step (iii) is not carried out) or step (iii) can be carried out by usual means, such as neutralisation, if necessary or desired, and isolation of the desired reaction products I.a and/or I.b (step (iv.1) by separation from the further components of the reaction mixture, such as unreacted compound II.a, acylating agent, base and acylation catalyst or undesired side products and, if desired, separation from each other. Separation can be carried out by usual means, such as extractive, distillative or chromatographic methods.

If compounds I.a or I.b are formed as different stereoisomers, these can be separated from each other if desired.

To obtain compounds I.a and/or I.b wherein R¹ is hydrogen, either the compounds I.a and/or I.b isolated according to step (iv.1) or the reaction mixture as obtained from step (ii) (if step (iii) is not carried out) or from step (iii) is hydrolized, e.g. by reaction with an acid or with a base. Hydrolysis is of course necessary (to obtain compounds I.a and/or I.b wherein R¹ is hydrogen) if in compounds II.a (and thus also in the resulting compounds I.a and/or I.b) R¹ is —C(O)R², but might also be necessary if in compounds II.a R¹ is hydrogen, since a part of the hydroxyl group might be acylated by the acylating agent, especially if this is used in excess.

If the reaction mixture as obtained from step (ii) (if step (iii) is not carried out) or from step (iii) is hydrolized (step (v.1)), the obtained reaction mixture can be worked-up and the desired reaction products I.a and/or I.b (step (iv.1) can be separated from the further components of the reaction mixture and also from each other by usual means, such as extraction, distillation or chromatographic methods.

The process of the invention offers a simple method for the preparation of compounds I.a and/or I.b starting from the readily available bulk chemicals isoprenol and isoprenol esters II.a. Compounds I.a and I.b can serve as intermediates in the preparation of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular of esters in which the OH group of retinol is esterified to —O—C(O)R²). Compounds I.a can be converted into compounds I.b by a known Pd-catalyzed double-bond isomerization reaction, as described e.g. in U.S. Pat. No. 4,124,619 or CN 103467287.

The invention relates moreover to the use of the compound of the formula I.a or I.b different from (E)-4-acetoxy-2-methylbut-2-enal or of a stereoisomer of the compound I.a or I.b different from (E)-4-acetoxy-2-methylbut-2-enal or of a mixture of different stereoisomers of the compound I.a and/or I.b or of a mixture of different compounds I.a and/or I.b as defined above as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²). As explained above, compounds I.a can be readily converted into I.b. The latter can be reacted in a Wittig reaction with a β-ionylidenethyltriphenylphosphonium salt to retinol (this results directly if in I.b R¹═H), stereoisomers thereof, esters thereof (esters in which the OH group of retinol is esterified to —O—C(O)R² result directly if in I.b R¹═C(O)R²) or stereoisomers of esters thereof, as described for example by J. Paust et al. in Carotenoids, Birkhäuser, 1996, vol. 2, pp. 258-292, in U.S. Pat. No. 5,087,762, by H. Ernst, Pure Appl. Chem. 2002, 74, 2213 or by G. L Parker et al. in Tetrahedron 2016, 72, 1645-1652. Other retinol derivatives are obtainable by usual means; e.g. by esterification of retinol (or stereoisomers thereof) with acids or acid derivatives different from R²—C(O)OH or derivatives thereof; or by oxidation of retinol (or stereoisomers thereof) to retinal (or stereoisomers thereof) or retinoic acid (or stereoisomers thereof). Retinol (or stereoisomers thereof) can be obtained by saponification (ester cleavage) of retinol esters (or stereoisomers thereof). These conversions are well known in the art.

The invention relates furthermore to a hydroperoxide compound of the formula III.a, III.b or III.c or a stereoisomer of the compound of the formula III.a, III.b or III.c or a mixture of different stereoisomers of the compound III.a, and/or III.b and/or III.c or a mixture of different compounds III.a, III.b and/or III.c

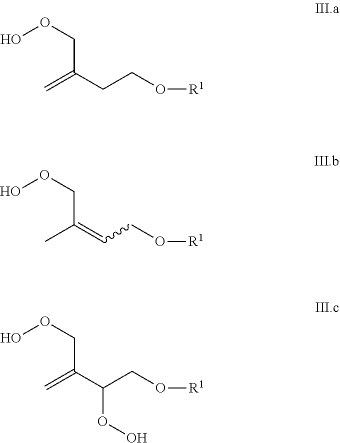

where
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl.

The hydroperoxides III.a, III.b and III.c are formed in step (ii) of the method of the invention. If the reaction mixture provided in step (i) does not contain an acylating agent, the hydroperoxides formed in step (ii) can be detected and also isolated, since in the absence of acylating agents their further reaction/decomposition is rather slow.

The invention relates preferably to a hydroperoxide compound of the formula III.a or III.b or a stereoisomer of the compound of the formula III.a or III.b or a mixture of different stereoisomers of the compound III.a and/or III.b or a mixture of different compounds III.a and/or III.b.

The invention relates alternatively preferably to a hydroperoxide compound of the formula III.a or III.c or a stereoisomer of the compound of the formula III.a or III.c or a mixture of different stereoisomers of the compound III.a and/or III.c or a mixture of different compounds III.a and/or III.c.

The invention relates in particular to a hydroperoxide compound of the formula III.a or a stereoisomer of the compound of the formula III.a or a mixture of different stereoisomers of the compound III.a or a mixture of different compounds III.a.

The invention relates furthermore to the use of the hydroperoxide compound of the formula III.a, III.b or III.c or of a stereoisomer of the compound of the formula III.a, III.b or III.c or of a mixture of different stereoisomers of the compound III.a, III.b and/or III.c or of a mixture of different compounds III.a, III.b and/or III.c as defined above, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²).

The conversion of the compounds III.a and/or III.b into compounds I.a and/or I.b is carried out by subjecting compounds III.a and/or III.b to step (iii) and optionally steps (iv) and (v) of the method of the invention described above. Compounds I.a and/or I.b can be converted into retinol, stereoisomers thereof, derivatives thereof or stereoisomers of derivatives thereof as described above.

The invention relates preferably to the use of the hydroperoxide compound of the formula III.a or III.b or of a stereoisomer of the compound of the formula III.a or III.b or of a mixture of different stereoisomers of the compound III.a and/or III.b or of a mixture of different compounds III.a and/or III.b as defined above, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²).

The invention relates in particular to the use of a hydroperoxide compound of the formula III.a or a stereoisomer of the compound of the formula III.a or a mixture of different stereoisomers of the compound III.a or a mixture of different compounds III.a as intermediate in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²).

The following examples serve as further illustration of the invention.

### EXAMPLES

The compounds were characterized by 1H-NMR and partially also by 13C-NMR. The NMR analysis was carried out on a Bruker 400 MHZ, 500 MHz and 700 MHz Spectrometer in CDCl₃. The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singlett.

### 1. Photooxidation of Isoprenyl Acetate and Simultaneous Kornblum-DeLaMare Rearrangement in a Double Jacketed Reactor

### Examples 1 to 4

Apparatus: Double jacket vessel, cylindrical, with tempered outer jacket, inner diameter 45 mm, total volume 150 mL (reaction volume approx. 24 mL, corresponds to approx. 18 mm filling height), illuminated from below by 24 LEDs with a wavelength of 405 nm, total radiometric power 27 W, impeller stirrer.

In a 50 mL glass vial, a mixture of 12.46 g (97.2 mmol, 1 eq) of isoprenyl acetate (IPA; compound II.a wherein R¹═C(O)CH₃), 6.15 g (77.7 mmol, 0.8 eq) of pyridine and 3.96 g (38.8 mmol, 0.4 eq) of acetic anhydride was prepared, and 0.68 g (3.5 mmol, 0.036 eq) of dimethyl phthalate as an internal NMR standard, 0.21 g (1.75 mmol, 0.018 eq) of N,N-dimethylaminopyridine (DMAP) (example 4:0.009 eq DMAP) and 4.4 μmol (0.000045 eq) of a photosensitizer (examples 1, 2 and 4: tetraphenylporphyrin (TPP), example 3 zinc tetraphenylporphyrin (Zn-TPP), example 4:0.000067 eq TPP) were dissolved in said mixture. The reaction mixture was poured into the temperature controlled double jacketed vessel and stirred at 1000 rpm. At the temperature indicated in the below table, the solution was irradiated from below for a period of 6 hours while 2 L/h of oxygen were introduced into the solution. During the reaction, up to 34 μmol of photosensitizer were added in portions. The reaction was terminated distinctly before complete conversion of isoprenyl acetate (the conversion rate for each experiment is listed in Table 1). At the end of the experiment, the reaction mixture was analysed without further work-up. No hydroperoxides were detected. The results are summarized in Table 1.

TABLE 1

| | | | |
|---|---|---|---|
| T | Conversion | Yield I.a^{#} | Yield I.b^{##} |

| No. | [° C.] | IPA [%] | [g] | [%]¹ | [g] | [%]¹ |
|---|---|---|---|---|---|---|
| 1 | 5 | 21.1 | 1.8 | 12.8 | 0.6 | 4.6 |
| 2 | 20 | 28.3 | 2.3 | 16.3 | 0.8 | 6.1 |
| 3* | 10 | 11.7 | 0.8 | 5.8 | 0.4 | 2.6 |
| 4** | 10 | 23.1 | 2.0 | 14.6 | 0.7 | 5.2 |

¹Yield relative to the amount of IPA as used in the reaction (here 97.2 mmol)
*Zn-TPP instead of TPP
**0.009 eq of DMPA; 0.000067 eq of TPP
^{#}Compound I.a wherein R¹ = C(O)CH₃
^{##}Mixture of E and Z isomers of compound I.b wherein R¹ = C(O)CH₃
I.a^{#}: ¹H-NMR (400 MHz, CDCl₃): δ = 9.56 (1H), 6.11 (1H), 6.36 (1H), 4.19 (2H), 2.61 (2H), 2.03 (3H)
I.b^{##}: ¹H-NMR (400 MHz, CDCl₃): δ = 9.56 (1H), 6.10 (1H), 4.60 (2H), 2.03 (3H), 1.84 (3H)

### Example 5

Apparatus: Like in examples 1-4, however illuminated from below by 24 LEDs with a wavelength of 465 nm, total radiometric power 31 W.

In a 50 mL glass bottle, a mixture of 12.57 g (98 mmol, 1 eq) of isoprenyl acetate (IPA), 6.21 g (78.5 mmol, 0.8 eq) of pyridine and 4.0 g (39.2 mmol, 0.4 eq) of acetic anhydride was prepared, and 0.68 g (3.5 mmol, 0.036 eq) of dimethyl phthalate as an internal NMR standard, 0.1 g (0.87 mmol, 0.009 eq) of N,N-dimethylaminopyridine (DMAP) and 6.7 mg (7.8 μmol, 0.00008 eq) of tris-(2,2′-bipyridine)-ruthenium bis(hexafluorophosphate) as a photosensitizer were dissolved in said mixture. The reaction solution was poured into the temperature controlled double jacket vessel and stirred at 1000 rpm. At 12° C., the solution was irradiated from below for a period of 6 hours while 2 L/h of oxygen were introduced into the solution. During the reaction, a further 3.9 mg (4.5 μmol, 0.000046 eq) of photosensitizer were added. The reaction was terminated distinctly before complete conversion of isoprenyl acetate (the conversion rate for the experiment is listed in Table 2). At the end of the experiment, the reaction mixture was analysed without further workup. No hydroperoxides were detected. The results are summarized in Table 2.

TABLE 2

| | | | |
|---|---|---|---|
| T | Conversion | Yield I.a^{#} | Yield I.b^{##} |

| No. | [° C.] | IPA [%] | [g] | [%]¹ | [g] | [%]¹ |
|---|---|---|---|---|---|---|
| 5 | 12 | 17.5 | 1.5 | 10.6 | 0.6 | 4.1 |

¹Yield relative to the amount of IPA as used in the reaction (here 98 mmol)

### Example 6

### Apparatus: Like in Examples 1-4

In a 50 mL glass vial, a mixture of 7.1 g (55.4 mmol, 1 eq) isoprenyl acetate (IPA), 6.11 g (77.2 mmol, 1.4 eq) of pyridine and 3.94 g (38.6 mmol, 0.7 eq) of acetic anhydride was prepared, and 0.69 g (3.55 mmol, 0.064 eq) of dimethyl phthalate as internal NMR standard, 8.42 g (54.7 mmol, 0.99 eq) of carbon tetrachloride, 0.11 g (0.89 mmol, 0.016 eq) of N,N-dimethylaminopyridine and 13.3 mg (21.6 μmol, 0.00039 eq) of tetraphenylporphyrin as photosensitizer were dissolved in said mixture. The reaction solution was poured into the temperature controlled double jacket vessel and stirred at 1000 rpm. At 32° C., the solution was irradiated from below for a period of 6 hours while 1.5 L/h of oxygen was introduced into the solution. During the reaction, a further 32.8 mg (53.4 μmol, 0.00096 eq) of photosensitizer were added in portions. The reaction was terminated distinctly before complete conversion of isoprenyl acetate (the conversion rate for the experiment is listed in Table 3). At the end of the experiment, the reaction mixture was analysed without further workup. No hydroperoxides were detected. The results are summarized in Table 3.

TABLE 3

| | | | |
|---|---|---|---|
| T | Conversion | Yield¹ I.a^{#} | Yield¹ I.b^{##} |

| No. | [° C.] | IPA [%] | [g] | [%]¹ | [g] | [%]¹ |
|---|---|---|---|---|---|---|
| 6 | 32 | 46.0 | 2.0 | 25.6 | 0.8 | 9.8 |

¹Yield relative to the amount of IPA as used in the reaction (here 55.4 mmol)

### 2. Photooxidation of Isoprenyl Acetate and Simultaneous Kornblum-DeLaMare Rearrangement in a Corning® G1 Photoreactor

Apparatus: Corning® G1 reactor (5 tempered G1 plates, layer thickness approx. 1 mm, each irradiated on both sides by LEDs, a total of 200 LEDs with a wavelength of 405 nm, total radiometric power 195 W), 100 mL miniplant reactor as feed vessel, impeller stirrer, gear pump.

### Examples 7-9

In the 100 mL miniplant reactor, a mixture of 83.0 g (647.5 mmol, 1 eq) of isoprenyl acetate (IPA), 40.73 g (515 mmol, 0.8 eq) of pyridine and 26.28 g (259 mmol, 0.4 eq) of acetic anhydride was prepared, and 4.55 g (23.3 mmol, 0.036 eq) of dimethyl phthalate as an internal NMR standard, 2.86 g (11.66 mmol, 0.018 eq) N,N-dimethylaminopyridine (DMAP) and 18 mg (29.1 μmol, 0.000045 eq) of tetraphenylporphyrin as a photosensitizer were dissolved in said mixture. The reaction solution was stirred at 100 rpm and pumped over the Corning® reactor in a circuit. At the temperature and pressure indicated in Table 6, the solution was irradiated for a period of 6 hours while 3 L/h of oxygen (example 8: air; example 9 mixture of oxygen:nitrogen=1:1) were introduced into the Corning® reactor. During the reaction, up to 74 mg (120 μmol) of further tetraphenylporphyrin were added in portions. The reaction was terminated distinctly before complete conversion of isoprenyl acetate (the conversion rate for each of the experiments is listed in Table 4). At the end of the experiment, the reaction mixture was analysed without further workup. No hydroperoxides were detected. The results are summarized in Table 4.

TABLE 4

| | T | p | Conversion | Yield¹ I.a^{#} | | Yield¹ I.b^{##} | |
|---|---|---|---|---|---|---|---|
| No. | [° C.] | [bar] | IPA [%] | [g] | [%]¹ | [g] | [%]¹ |
| 7 | 30 | 2.6-3.1 | 11.5 | 5.8 | 6.3 | 3.1 | 3.4 |
| 8* | 30 | 1.8-2.8 | 15.5 | 5.6 | 6.1 | 2.9 | 3.2 |
| 9** | 30 | 1.9-2.3 | 16.7 | 5.6 | 6.1 | 3.0 | 3.3 |

¹Yield relative to the amount of IPA as used in the reaction (here 647.5 mmol)
*air instead of oxygen
**mixture of oxygen and nitrogen in a mixing ratio of 1:1

### Example 10

In the 100 mL miniplant reactor, a mixture of 56.5 g (440.8 mmol, 1 eq) of isoprenyl acetate (IPA), 40.73 g (515 mmol, 1.16 eq) of pyridine and 26.28 g (257 mmol, 0.58 eq) of acetic anhydride was prepared, and 4.55 g (23.4 mmol, 0.053 eq) of dimethyl phthalate as internal NMR standard, 26.5 g (222 mmol, 0.5 eq) of chloroform, 2.86 g (23.41 mmol, 0.053 eq) of N,N-dimethylaminopyridine and 18.2 mg (29.6 μmol, 0.000067 eq) of tetraphenylporphyrin as photosensitizer were dissolved in said mixture.

The reaction solution was stirred at 100 rpm and pumped over the Corning® reactor in a circuit. At 30° C., the solution was irradiated for a period of 6 hours while 3 L/h of oxygen were introduced into the Corning® reactor at 1.9-2.4 bar. During the reaction, a further 26.2 mg (42.8 μmol) of tetraphenylporphyrin were added in portions. The reaction was terminated distinctly before complete conversion of isoprenyl acetate (the conversion rate for the experiment is listed in Table 5). At the end of the experiment, the reaction mixture was analysed without further workup. No hydroperoxides were detected. The results are summarized in Table 5.

TABLE 5

| | | | |
|---|---|---|---|
| T | Conversion | Yield¹ I.a^{#} | Yield¹ I.b^{##} |

| No. | [° C.] | IPA [%] | [g] | [%]¹ | [g] | [%]¹ |
|---|---|---|---|---|---|---|
| 10 | 30 | 27.0 | 7.1 | 11.3 | 3.5 | 5.7 |

¹Yield relative to the amount of IPA as used in the reaction (here 440.8 mmol)

### Example 11

In the 100 mL miniplant reactor, a mixture of 43.25 g (337.4 mmol, 1 eq) of isoprenyl acetate (IPA), 40.73 g (515 mmol, 1.52 eq) of pyridine and 26.29 g (257.5 mmol, 0.76 eq) of acetic anhydride was prepared, and 4.55 g (23.4 mmol, 0.069 eq) of dimethyl phthalate as internal NMR standard, 39.74 g (332.9 mmol, 0.99 eq) chloroform, 2.86 g (23.41 mmol, 0.069 eq) of N,N-dimethylaminopyridine and 18.2 mg (29.6 μmol, 0.000088 eq) of tetraphenylporphyrin as photosensitizer were dissolved in said mixture. The reaction solution was stirred at 100 rpm and pumped over the Corning® reactor in a circuit. At 50° C., the solution was irradiated for a period of 6 hours while 3 L/h of oxygen were introduced into the Corning® reactor at 1.9-2.3 bar. During the reaction, a further 20.3 mg (33.0 μmol) of tetraphenylporphyrin were added in portions. The reaction was terminated distinctly before complete conversion of isoprenyl acetate (the conversion rate for the experiment is listed in Table 6). At the end of the experiment, the reaction mixture was analysed without further workup. No hydroperoxides were detected. The results are summarized in Table 6.

TABLE 6

| | | | |
|---|---|---|---|
| T | Conversion | Yield¹ I.a^{#} | Yield¹ I.b^{##} |

| No. | [° C.] | IPA [%] | [g] | [%]¹ | [g] | [%]¹ |
|---|---|---|---|---|---|---|
| 11 | 50 | 22.5 | 4.6 | 9.6 | 2.6 | 5.4 |

¹Yield relative to the amount of IPA as used in the reaction (here 337.4 mmol)

### 3. Photooxidation of Isoprenyl Acetate and Simultaneous Kornblum-DeLaMare Rearrangement in a Corning® G3 Photoreactor

Apparatus: Corning® G3 reactor (tempered G3 plate, layer thickness approx. 1 mm, each irradiated on both sides by LEDs, a total of 384 LEDs with a wavelength of 470 nm, total radiometric power 576 W), feed vessel, gear pump.

### Example 12

In a 500 mL glass vial, a mixture of 155 g (1209 mmol, 1 eq) of isoprenyl acetate (IPa), 76.6 g (0.8 eq) of pyridine and 49.4 g (0.4 eq) of acetic anhydride was prepared, and 8.22 g (0.035 eq) of dimethyl phthalate as an internal NMR standard, 1.33 g (0.009 eq) of N, N-dimethylaminopyridine and 79 mg (0.000076 eq) of tris-(2,2′-bipyridine)ruthenium bis-(hexafluorophosphate) as a photosensitizer were dissolved in said mixture. The reaction solution was poured into a feed vessel, then pumped into the Corning® reactor and circulated at 1980 mL/min. The solution was irradiated at 18° C. for a period of 6 hours while 3 L/h of oxygen were introduced into the Corning® reactor at 5 bar. The reaction was terminated distinctly before complete conversion of isoprenyl acetate (the conversion rate for the experiment is listed in Table 7). At the end of the experiment, the reaction mixture was analysed without further workup. No hydroperoxides were detected. The results are summarized in Table 7.

TABLE 7

| | T | Conversion | Yield¹ I.a^{#} | | Yield¹ I.b^{##} | |
|---|---|---|---|---|---|---|
| No. | [° C.] | IPA [%] | [g] | [%]¹ | [g] | [%]¹ |
| 12 | 18 | 10.2 | 11.8 | 6.9 | 4.6 | 2.7 |

¹Yield relative to the amount of IPA as used in the reaction (here 1209 mmol)

### Example 13

In a 500 mL glass vial, a mixture of 103.0 g (803.6 mmol, 1 eq) of isoprenyl acetate (IPA), 50.8 g (642.9 mmol, 0.8 eq) of pyridine and 32.8 g (32.1 mmol, 0.4 eq) of acetic anhydride was prepared, and 5.48 g (28.2 mmol, 0.035 eq) of dimethyl phthalate an internal NMR standard, 0.88 g (7.24 mmol, 0.009 eq) of N,N-dimethylaminopyridine and 105.1 mg (122.3 μmol, 0.00015 eq) of tris-(2,2′-bipyridine)-ruthenium bis(hexafluorophosphate) as a photosensitizer were dissolved in said mixture. The reaction solution was poured into a feed vessel and then pumped into the Corning® reactor and circulated at 1760 mL/min. The solution was irradiated at 18° C. for a period of 6 hours while 3 L/h of oxygen were introduced into the Corning® reactor at 5 bar. The reaction was terminated distinctly before complete conversion of isoprenyl acetate (the conversion rate for the experiment is listed in Table 8). At the end of the experiment, the reaction mixture was analysed without further workup. No hydroperoxides were detected. The results are summarized in Table 8.

TABLE 8

| | | | |
|---|---|---|---|
| T | Conversion | Yield I.a^{#} | Yield I.b^{##} |

| No. | [° C.] | IPA [%] | [g] | [%]¹ | [g] | [%]¹ |
|---|---|---|---|---|---|---|
| 13 | 18 | 24.2 | 17.2 | 15.0 | 6.4 | 5.6 |

¹Yield relative to the amount of IPA as used in the reaction (here 803.6 mmol)

### 4. Photooxidation of Isoprenyl Acetate to Isoprenyl Acetate Hydroperoxide

### Example 14

Apparatus: Corning® G1 photoreactor (5 tempered G1 plates, layer thickness approx. 1 mm, each irradiated on both sides by LEDs, a total of 200 LEDs with a wavelength of 405 nm, total radiometric power 195 W), 100 mL miniplant reactor, impeller stirrer, gear pump.

In the 100 mL miniplant reactor, a mixture of 135.0 g (1053.3 mmol, 1 eq) of isoprenyl acetate (IPA) and 6.22 g (32.0 mmol, 0.03 eq) of dimethyl phthalate as internal NMR standard was prepared and 15 mg (24.7 μmol, 0.000023 eq) of tetraphenylporphyrin as photosensitizer was dissolved in said mixture. The reaction solution was stirred at 100 rpm and pumped over the Corning® reactor in a circuit. At the temperature indicated in Table 9, the solution was irradiated for a period of 6 hours while 3 L/h of oxygen were introduced into the Corning® reactor at 1.7-2.4 bar. The reaction was terminated distinctly before complete conversion of isoprenyl acetate (the conversion rate for the experiment is listed in Table 9). At the end of the experiment, the reaction mixture was analysed without further workup. The results are summarized in Table 9.

TABLE 9

| | | | Yield¹ hydroperoxide⁺ | |
|---|---|---|---|---|
| No. | T [° C.] | Conversion IPA [%] | [g] | [%]¹ |
| 14 | 30 | 29.7 | 24.6 | 14.6 |

¹Yield relative to the amount of IPA as used in the reaction (here 1053.3 mmol)
⁺compound III.a wherein R¹ = —C(O)CH₃

III.a⁺: ¹H-NMR (500 MHz, CDCl₃): δ=5.19 (1H), 5.10 (1H), 4.47 (2H), 4.26 (2H), 2.47 (2H), 2.06 (3H)

¹³C-NMR (125 MHZ, CDCl₃): δ=171.19 (s), 140.75 (s), 116.58 (t), 79.82 (t), 62.69 (t), 32.36 (t), 22.34 (q)

Moreover, compound III.c wherein R¹═C(O)CH₃ was identified, which is presumed to be the result of the further photooxidation of compound III.b taking place in competition with the conversion of III.b to I.b, or directly of (double) photooxidation of II.a:

III.c⁺: ¹H-NMR (700 MHZ, CDCl₃): δ=5.45 (1H), 5.41 (1H), 4.71 (1H), 4.55 (2H), 4.39 (2H), 2.09 (3H)

¹³C-NMR (175 MHZ, CDCl₃): δ=170.6 (s), 139.8 (s), 120.2 (t), 83.3 (d), 77.8 (t), 62.7 (t), 21.1 (q)

### 5. Photooxidation of Isoprenol to Isoprenol Hydroperoxide

### Example 15

Apparatus: Corning® G1 photoreactor (5 tempered G1 plates, layer thickness approx. 1 mm, each irradiated on both sides by LEDs, a total of 200 LEDs with a wavelength of 610 nm, total radiometric power 83 W), 100 mL miniplant reactor, impeller stirrer, gear pump.

In the 100 mL miniplant reactor, a mixture of 135.0 g (1567.4 mmol, 1 eq) of isoprenol (IP; compound II.a wherein R¹ is H) and 9.13 g (47.0 mmol, 0.03 eq) of dimethyl phthalate as the internal NMR standard was prepared, and 28.3 mg (84 μmol, 0.000054 eq) of methylene blue monohydrate as a photosensitizer was dissolved in said mixture. The reaction solution was stirred at 300 rpm and pumped over the Corning® reactor at 100 mL/min in a circuit. At 30° C., the solution was irradiated for a period of 4 hours while 3 L/h of oxygen were introduced into the Corning® reactor at 5 bar. During the reaction, a further 63.5 mg (188 μmol) of photosensitizer were added in portions The reaction was terminated distinctly before complete conversion of isoprenol (the conversion rate for the experiment is listed in Table 10). At the end of the experiment, the reaction mixture was analysed without further workup. The results are summarized in Table 10.

TABLE 10

| | T | Conversion | Yield¹ hydroperoxide⁺⁺ | | Yield¹ I.a^{###} | |
|---|---|---|---|---|---|---|
| No. | [° C.] | IP [%] | [g] | [%]¹ | [g] | [%]¹ |
| 15 | 30 | 11.7 | 7.3 | 3.9 | 0.1 | 0.1 |

¹Yield relative to the amount of IP as used in the reaction (here 1567.4 mmol)
⁺⁺compound III.a wherein R¹ = H
^{###}Compound I.a wherein R¹ = H
III.a⁺⁺: ¹H-NMR (400 MHz, CDCl₃): δ = 5.24 (1H), 5.16 (1H), 4.47 (2H), 3.86 (2H), 2.41 (2H)

III.a⁺⁺: 1H-NMR (400 MHZ, CDCl₃): δ=5.24 (1H), 5.16 (1H), 4.47 (2H), 3.86 (2H), 2.41 (2H) ¹ Yield relative to the amount of IP as used in the reaction (here 1567.4 mmol)+⁺⁺ compound III.a wherein R¹═H^{###} Compound I.a wherein R¹═H

### Example 16

Apparatus: Double jacket vessel, cylindrical, with tempered outer jacket, inner diameter 45 mm, total volume 150 mL (reaction volume approx. 38 mL, corresponds to approx. 24 mm filling height), illuminated from below by 24 LEDs with a wavelength of 405 nm, total radiometric power 27 W, impeller stirrer.

In a 50 mL glass vial, a mixture of 30.0 g (348.3 mmol, 1 eq) of isoprenol (IP), 1.39 g (7.2 mmol, 0.021 eq) of dimethyl phthalate as internal NMR standard and 1.5 g (12.5 mmol, 0.036 eq) of chloroform was prepared, and 4.6 mg (7.5 μmol, 0.000021 eq) of tetraphenylporphyrin as photosensitizer was dissolved in said mixture. The reaction solution was poured into the temperature controlled double jacket vessel and stirred at 800 rpm. At 10° C., the solution was irradiated for a period of 5 hours while 2 L/h of oxygen were introduced into the solution. During the reaction, a further 8.2 mg (13.3 μmol) of photosensitizer were added in portions. The reaction was terminated distinctly before complete conversion of isoprenol (the conversion rate for the experiment is listed in Table 11). At the end of the experiment, the reaction mixture was analysed without further workup. The results are summarized in Table 11.

TABLE 11

| | T | Conversion IP | Yield¹ hydroperoxide⁺⁺ | | Yield¹ I.a^{#} | |
|---|---|---|---|---|---|---|
| No. | [° C.] | [%] | [g] | [%]¹ | [g] | [%]¹ |
| 16 | 30 | 23.0 | 3.6 | 8.6 | 0.1 | 0.3 |

¹Yield relative to the amount of IP as used in the reaction (here 348.3 mmol)

### 6. Kornblum-DeLaMare Rearrangement of the Hydroperoxide III.a Wherein R¹═H

### Example 17

Apparatus: 0.25 l miniplant reactor with impeller stirrer, 4-fold baffle, ministat, dropping funnel.

10 g of a solution of 1.81 g (15.32 mmol) of isoprenol hydroperoxide (compound III.a wherein R¹═H) as obtained in example 15 in isoprenol was placed in a 250 ml miniplant reactor, 133 g of dichloromethane were added and then 33.48 g (423.26 mmol) of pyridine were added dropwise at 17° C. in 10 min. After cooling to 3° C., 17.28 g (169.26 mmol) of acetic anhydride were added in 20 min. The reaction mixture was stirred at 0° C. for 17.25 h, and then 50 g of water were added in 30 min. The yellow-brown emulsion was heated to 22° C., the phases were separated, and the organic phase was extracted once with 10% HCl, once with saturated NaHCO₃ solution, and once with water. Then the red-brown organic phase was concentrated on the rotary evaporator at 40° C./30 mbar. 8.98 g of a solution of 1.68 g (11.8 mmol, 77%) of the compound I.a wherein R¹ is H in isoprenylacetate were obtained.

The yields given above are relative to the amount of the starting compound (IPA or IP) as used in the reaction. Since the reactions are carried out so that only a rather small portion of the starting compound is reacted (and the remainder can further serve as dispersing medium), only yields based on the amount of the reacted starting material reflect the effectiveness of the reaction. These can be calculated by relating the yields of I.a and I.b to the converted amount of IPA or IP (not shown in the above tables).

1.-20. (canceled)

21. A method for preparing a compound of formula I.a or of formula I.b or a mixture thereof or a stereoisomer of the compound I.a or I.b or a mixture of different stereoisomers of the compound I.a and/or I.b or a mixture of different compounds I.a and/or I.b 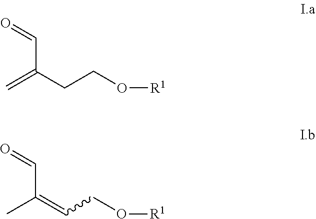
wherein
R¹ is hydrogen or —C(═O)R²; and
R² is C₁-C₂₀-alkyl,
the method comprising:
(i) providing a reaction mixture comprising a compound of the formula II.a 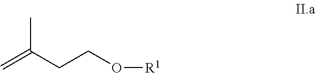
wherein R¹ is as defined above;
a photosensitizer and optionally an acylating agent;
(ii) passing an oxygen-containing gas through the reaction mixture provided in step (i) and simultaneously irradiating the reaction mixture with light;
(iii) if in step (i) no acylating agent has been provided, adding an acylating agent to the reaction mixture obtained in step (ii);
(iv.1) optionally, after completion of the reaction, isolating the one or more compounds (I.a) or (I.b) obtained in step (ii) or (iii); and
(v.1) optionally hydrolysing the one or more compounds (I.a) or (I.b) isolated in step (iv.1) to compounds (I.a) or (I.b) wherein R¹ is hydrogen;
or
(iv.2) optionally, hydrolysing the reaction mixture obtained in step (ii) or (iii); and
(v.2) optionally, isolating the one or more compounds (I.a) or (I.b) obtained in step (iv.2).

22. The method according to claim 21, wherein R² is C₁-C₄-alkyl or n-C₁₅-alkyl.

23. The method according to claim 22, wherein R² is C₁-C₄-alkyl.

24. The method according to claim 21, wherein the acylating agent used in step (i) or (iii) is selected from the group consisting of carboxylic halides,
R^{2a}—C(═O)—X, carboxylic acid anhydrides R^{2a}—C(═O)—O—C(═O)—R^{2a}, and ketenes R²ª—C(H)═C═O, wherein R^{2a} has independently one of the meanings given for R² in claim 21 and X is Cl, Br or I.

25. The method according to claim 24, wherein the acylating agent used in step (i) or (iii) is a carboxylic acid anhydride R^{2a}—C(═O)—O—C(═O)—R^{2a}, wherein R^{2a} is C₁-C₄-alkyl.

26. The method according to claim 21, wherein the photosensitizer is selected from the group consisting of fluorescein, eosin, rose bengal, erythrosine, tetraphenylporphyrin, cobalt-tetraphenylporphyrin, zinc-tetraphenylporphyrin, hematoporphyrin, rhodamine B, basacryl brilliant red, methyl violet, methylene blue, fullerene C₆₀, fullerene C₇₀, graphene, carbon nanotubes, Ru(bpy)₃²⁺ salts, Ru(phen)₃²⁺ salts, cercosporin, hypocrellin-A and mixtures thereof.

27. The method according to claim 26, wherein the photosensitizer is selected from the group consisting of tetraphenylporphyrin, cobalt-tetraphenylporphyrin, zinc-tetraphenylporphyrin, methylene blue, Ru(bpy)₃²⁺ salts and Ru(phen)₃²⁺ salts.

28. The method according to claim 21, wherein in step (ii) the reaction mixture is irradiated with light in the wavelength range of from 350 to 800 nm.

29. The method according to claim 27, wherein the photosensitizer is tetraphenylporphyrin, cobalt-tetraphenylporphyrin or zinc-tetraphenylporphyrin and in step (ii) the reaction mixture is irradiated with light in the wavelength range of from 400 to 430 nm; or the photosensitizer is methylene blue and in step (ii) the reaction mixture is irradiated with light in the wavelength range of from 600 to 620 nm, or the photosensitizer is a Ru(bpy)₃²⁺ salt or a Ru(phen)₃²⁺ salt and in step (ii) the reaction mixture is irradiated with light in the wavelength range of from 450 to 480 nm.

30. The method according to claim 21, wherein the oxygen-containing gas used in step (ii) is selected from the group consisting of oxygen, air and mixtures of oxygen and nitrogen containing oxygen in a range of from 1 to 99% by weight, relative to the total weight of the mixture.

31. The method according to claim 21, wherein step (ii) is carried out neat.

32. The method according to claim 21, wherein step (ii) is carried out in the presence of a chlorinated C₁-C₂-alkane, wherein the molar ratio of the compound (II.a) provided in step (i) to the chlorinated C₁-C₂-alkane is of from 10:1 to 1:1.5.

33. The method according to claim 21, wherein in case that in step (i) an acylating agent has been provided, step (ii) is carried out in the presence of a base and in case that in step (i) no acylating agent has been provided, in step (iii) also a base is added to the reaction mixture obtained in step (ii), wherein the base is selected from tertiary amines, basic 3- to 10-membered saturated, partially unsaturated or aromatic monocyclic or bicyclic heterocyclic rings containing 1, 2, 3 or 4 nitrogen atom as ring members, guanidines and amidines.

34. The method according to claim 33, wherein in case that in step (i) an acylating agent has been provided, step (ii) is carried out in the presence of a base which is selected from 5- to 10-membered monocyclic or bicyclic heteroaromatic rings containing 1 or 2 nitrogen ring atoms as ring members; wherein the base is selected from the group consisting of imidazole, pyridine, pyrazine, pyridazine, pyrimidine, quinolone and isoquinoline; wherein the monocyclic or bicyclic heteroaromatic rings are unsubstituted or carry 1, 2 or 3 C₁-C₄-alkyl substituents.

35. The method according to claim 34, wherein the base is pyridine which is unsubstituted or carries 1, 2 or 3 C₁-C₄-alkyl substituents.

36. The method according to claim 21, wherein in case that in step (i) an acylating agent has been provided, step (ii) is carried out in the presence of an acylation catalyst, and in case that in step (i) no acylating agent has been provided, in step (iii) also an acylation catalyst is added to the reaction mixture obtained in step (ii);
wherein the acylation catalyst is selected from the group consisting of 4-dimethylaminopyridine (DMAP), 4-pyrrolidinopyridine (PPY), 1,6-dibenzyl-2,3,5,6-tetrahydro-1H,4H-1,3a,6,8-tetraazaphenalene (Super DMAP) and acylation catalysts of the general formulae 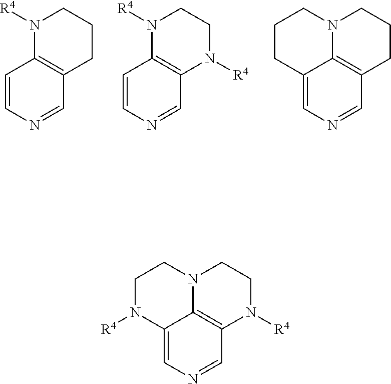
wherein each R⁴ is independently methyl or ethyl.

37. The method according to claim 36, wherein the acylation catalyst is 4-dimethylaminopyridine (DMAP).

38. The method according to claim 21, wherein in step (i) an acylating agent, a base and an acylation catalyst is provided and step (iii) is not carried out.

39. The method according to claim 33, wherein the base is selected from the group consisting of tertiary amines of the formula N(R³)₃, wherein each R³ is independently C₁-C₄-alkyl; 3- to 8-membered saturated monocyclic or bridged heterocyclic rings containing 1 or 2 nitrogen atoms or 1 nitrogen atom and one oxygen atom as ring members; in particular compounds of the formula NR³¹(R³²)₂, wherein R³¹ is hydrogen or C₁-C₄-alkyl and the two R³² form together a bridging group —(CH₂)ₙ—, wherein n is 2, 3, 4 or 5, or form together a bridging group —(CH₂)₂—N(R³³)—(CH₂)₂— or —(CH₂)₂—O—(CH₂)₂—, wherein R³³ is hydrogen or C₁-C₄-alkyl;
or DABCO; 5- to 10-membered monocyclic or bicyclic heteroaromatic rings containing 1 or 2 nitrogen ring atoms as ring members; in particular selected from the group consisting of imidazole, pyridine, pyrazine, pyridazine, pyrimidine, quinolone and isoquinoline; wherein the monocyclic or bicyclic heteroaromatic rings are unsubstituted or carry 1, 2 or 3 C₁-C₄-alkyl substituents; guanidines of the formula (R³)₂N—C(═NR³)—N(R³)₂, wherein each R³ is independently C₁-C₄-alkyl or two R³ bound to the same nitrogen atom or on different nitrogen atoms form together a bridging group —(CH₂)ₙ—, wherein n is 2, 3, 4 or 5; and the bicyclic amidines DBN or DBU.

40. A hydroperoxide compound of the formula III.a, III.b or III.c or a stereoisomer of the compound of the formula III.a, III.b or III.c or a mixture of different stereoisomers of the compound III.a, III.b and/or III.c or a mixture of different compounds III.a, III.b and/or III.c 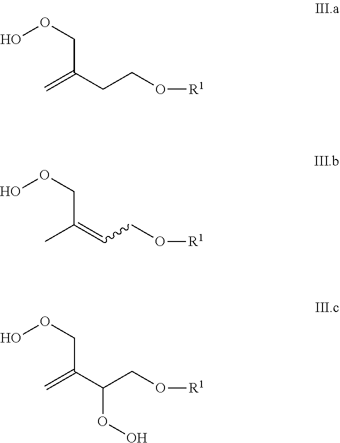
wherein
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl;
a hydroperoxide compound of the formula III.a or III.b or a stereoisomer of the compound of the formula III.a or III.b or a mixture of different stereoisomers of the compound III.a and/or III.b or a mixture of different compounds III.a and/or III.b.
